(19) United States
(12) Patent Application Publication
(10) Pub. No.: US 2016/0139123 A1
(43) Pub. Date: May 19, 2016

(54) Shear Wave Imaging Method and Installation for Collecting Information on a Soft Solid

(71) Applicants: INSTITUT NATIONAL DE LA SANTE ET DE LA RECHERCHE MEDICALE (INSERM), Paris (FR); CENTRE NATIONAL DE LA RECHERCHE SCIENTIFIQUE (C.N.R.S), Paris (FR); UNIVERSITE D'ANGERS, Paris (FR); UNIVERSITE DE NANTES, Nantes (FR); CHU NANTES, Nantes Cedex 1 (FR); UNIVERSITE PIERRE ET MARIE CURIE (PARIS 6), Paris (FR); ASSISTANCE PUBLIQUE - HOPITAUX DE PARIS, Paris (FR)

(72) Inventors: Stefan Catheline, Lyon (FR); Jean-Yves Chapelon, Lyon Cedex 03 (FR); Remi Souchon, Lyon (FR); Pol Grasland-Mongrain, Lyon Cedex 03 (FR); Cyril Lafon, Lyon (FR)

(21) Appl. No.: 14/904,300

(22) PCT Filed: Jul. 10, 2014
(86) PCT No.: PCT/EP2014/064928

§ 371 Date: Jan. 11, 2016

(30) Foreign Application Priority Data
Jul. 12, 2013 (EP) 13305995.6

(51) Int. Cl. G01N 33/569; A61K 39/02; A61K 35/17; A61K 35/74

(52) U.S. Cl. CPC G01N 33/56972; A61K 35/74; A61K 39/0208; A61K 35/17; A61K 2039/5154

(57) ABSTRACT

The invention relates to methods comprising a step consisting of determining the proportion and/or the level of T regulatory lymphocytes with a CD4⁺ CD8αα^{low} Foxp3^{neg} phenotype specific for Faecalibacterium prausnitzii for (i) diagnosing, (ii) prognosing outcome of, or (iii) predicting the risk of developing, an inflammatory bowel disease in a patient. The invention also concerns the treatment of an inflammatory bowel disease. The invention further relates to the kits that are useful in the above methods for diagnosing/prognosing an inflammatory bowel disease, and in the treatment of an inflammatory bowel disease. In a particular embodiment, the inflammatory bowel disease is the Crohn's disease.

The invention relates to methods comprising a step consisting of determining the proportion and/or the level of T regulatory lymphocytes with a CD4⁺ CD8αα^{low} Foxp3^{neg} phenotype specific for a microorganism of the gut flora, in particular T regulatory lymphocytes with a CD4⁺ CD8αα^{low} Foxp3^{neg} phenotype specific for Faecalibacterium prausnitzii, for (i) diagnosing, (ii) prognosing outcome of, or (iii) predicting the risk of developing, an inflammatory bowel disease in a patient.

The invention also concerns the treatment of an inflammatory bowel disease.

The invention further relates to the kits that are useful in the above methods for diagnosing/prognosing an inflammatory bowel disease, and in the treatment of an inflammatory bowel disease.

In a particular embodiment, the inflammatory bowel disease is the Crohn's disease.

### BACKGROUND OF THE INVENTION

The intestinal mucosa is constantly exposed to a high diversity of antigens originating from the food and commensal microbes. Therefore, to prevent inappropriate immune responses and maintain intestinal homeostasis, the gut immune system has evolved diverse regulatory pathways. Recent studies revealed that the bacteria of the gastrointestinal tract play a major role in shaping local and systemic immune responses (Honda, K. & Littman, D. R., Annu Rev Immunol 30, 759-795, 2012). One of the regulatory mechanisms identified in mice is the induction by specific bacteria of different effector and regulatory T cell subsets, whose adequate balance is required for the maintenance of gut homeostasis (Honda, K. & Littman, D. R., Annu Rev Immunol 30, 759-795, 2012).

The breakdown of any of the regulatory pathways of the gut immune system may lead to chronic inflammation, in particular inflammatory bowel disease (hereinafter abbreviated as “IBD”) (Maloy, K. J. & Powrie, F. Nature 474, 298-306, 2011).

Inflammatory bowel disease is a group of inflammatory conditions characterized by a chronic and relapsing inflammation of the gastrointestinal tract. The two most common forms of IBD are Crohn's disease and ulcerative colitis. While ulcerative colitis is limited to colon (large intestine) and rectum, Crohn's disease can involve any part of the gastrointestinal tract from mouth to anus, but it most commonly affects the small intestine and/or the colon. The manifestations of inflammatory bowel disease depend on the area of the intestinal tract involved (Baumgart and Sandbom, Lancet, 369: 1641-1657, 2007). The symptoms, however, are not specific for this disease. They include abdominal pain, vomiting, diarrhea, rectal bleeding, severe internal cramps/muscle spasms in the region of the pelvis, weight loss, anemia. Therefore, diagnosis is generally established by assessment of inflammatory markers in blood coupled with colonoscopy and biopsy of pathological lesions.

The pathogenesis involves an inappropriate and ongoing activation of the mucosal immune system driven by the presence of the intestinal microbiota. IBD is characterized by a reduced diversity of Bacteroidetes and Firmicutes (especially the Clostridia class), the two most abundant phyla in human faeces. Notably, the Faecalibacterium prausnitzii level appears to be consistently reduced in the intestinal microbiota of Crohn's disease patients, particularly those with ileal involvement (Sokol, H., et al., Proc Natl Acad Sci USA 105, 16731-16736, 2008; Willing, B., et al., Inflamm Bowel Dis 15, 653-660, 2009).

One of the regulatory pathways involved in intestinal homeostasis relies on the accumulation or local induction of regulatory T cells (Treg) in the gut mucosa (Tanoue, T. & Honda, K., Semin Immunol 24, 50-57, 2012). CD4 T cells that express the transcription factor fork head box p3 (Foxp3) are the best-known Treg. Among these cells, two subtypes can be distinguished. Natural Treg differentiate in the thymus in response to self-antigens and prevent self-reactive immune responses (Sakaguchi, et al., Cell 133, 775-787, 2008). Induced Treg (iTreg) differentiate in the periphery from conventional CD4 T cells under various conditions including chronic challenge by non-self-antigens such as commensal bacteria and are strong contributors to tissue homeostasis (Bilate, A. M. & Lafaille, J. J., Annu Rev Immunol 30, 733-758, 2012). In mice, Foxp3 iTreg cells are abundant in the intestinal mucosa, particularly in the colon, where they play a key role in the prevention of colitis (Atarashi, K., et al., Science 331, 337-341, 2011). Recent studies demonstrated that a cocktail of indigenous Clostridium bacteria from the gut microbiota, is a strong inducer of Foxp3 iTreg in the mouse colonic lamina propria (LP) (Atarashi, K., et al., Science 331, 337-341, 2011). Foxp3 Treg are also present in the human gut mucosa, but their exact origin, distribution and contribution to IBD prevention still remain to be elucidated. Indeed, Foxp3 Treg inactivation, due to Foxp3 mutations, is not always associated with colitis, and there are a greater number of Foxp3 cells with seemingly normal suppressive capacity in the inflamed mucosa of IBD patients than in the normal mucosa of healthy donors (Buckner, J. H., Nat Rev Immunol 10, 849-859, 2010). Therefore, it has been postulated that other Treg or other suppressive mechanisms may regulate colon immune homeostasis in humans. Further, the existence and potential role of microbiota-induced Treg in humans remain to be addressed.

Nowadays, there is a great interest in identifying the cells or molecular mechanisms involved in maintaining colon immune homeostasis in the human and responsible for IBD prevention. Indeed, these cells/mechanisms would be valuable biomarkers useful for IBD diagnosis and prognosis, in particular Crohn's disease, and would be useful to develop therapeutic strategies for treating IBD.

### DESCRIPTION OF THE INVENTION

In an attempt to find the cells and/or the mechanisms responsible for intestinal homeostasis in human, the inventors have identified in the human colonic mucosa a new subset of T regulatory lymphocytes, i.e. a subset of Foxp3^{neg} IL-10-secreting T regulatory lymphocytes characterised by CD4⁺ CD8αα^{low} phenotype (hereinafter abbreviated as “DP8α Treg”). In particular, the inventors have identified DP8α Treg, the repertoire of which is skewed toward the recognition of Faecalibacterium prausnitzii (hereinafter abbreviated as “F. prausnitzii”), a major bacterium in the human microbiota.

CD4⁺CD8⁺ T cells were first identified in the small intestine mucosa of healthy subjects by Abuzakouk et al. (Eur J Gastroenterol Hepatol 10, 325-329, 1998). Recently, a study reported that these T cells are decreased in patients afflicted with celiac disease, an autoimmune disorder of the small intestine that occurs in genetically predisposed people (Carton et al., Eur J Gastroenterol Hepatol 16, 961-968, 2004). However, to date none of these studies discloses or even suggests that CD4⁺ CD8αα^{low} T cells are a subset of T lymphocytes with a regulatory activity, let alone that F. prausnitzii activated-CD4⁺ CD8αα^{low} Treg are involved in intestinal homeostasis.

Further, the inventors have unexpectedly found that these DP8α T regulatory lymphocytes (hereinafter abbreviated as “Treg”) can circulate from the colon to the blood, so that they can be quantified in colonic mucosa as well as directly in blood.

Although CD4⁺ CD8αα^{low} Foxp3^{neg} PBL (peripheral blood lymphocytes) lacked Treg markers ex vivo, the inventors found that a fraction of these cells reacted specifically to F. prausnitzii and that all acquired Treg markers and functions after sorting and in vitro expansion.

The inventors have also found that the frequency of F. prausnitzii-induced DP8α T regulatory lymphocytes is decreased in the blood and inflamed mucosa of inflammatory bowel disease (IBD) patients, when compared to healthy subjects.

Therefore, this newly described F. prausnitzii-induced DP8αTreg provides a new reliable biomarker for diagnosing, prognosing IBD, and for predicting whether a subject is at risk developing an IBD.

Besides, discovery that F. prausnitzii-induced DP8αTreg play a key role in gut homeostasis also provides new therapeutic strategies for treating IBD. In particular, F. prausnitzii-induced DP8αTreg, thanks to their particular characteristics, could be useful for adoptive transfer purposes in the context of IBD treatment. Indeed, the inventors have shown that, in contrast with most Treg cells, DP8αTreg proliferated under T-cell receptor (hereinafter abbreviated as “TCR”) stimulation in vitro (not anergic) (i.e. incubation of DP8αTreg with antigen-presenting cells loaded with F. prausnitzii induced proliferation of these Treg). Further, the inventors showed that cell lines derived from DP8αTreg have a high phenotypic stability, together with an in vitro-proliferative capacity.

Further, effectiveness of preventive or therapeutic treatments of IBD, (notably immunosuppressant, biologics or probiotics), can be assessed by determining the level of F. prausnitzii-induced DP8αTreg in a biological sample (e.g. blood and colonic mucosa) of a treated subject. Moreover, DP8αTreg level in a biological sample might also be used as a biomarker to predict relapse and thus anticipate treatment modifications.

### DEFINITIONS

As used herein, “biological sample” may consist of a colonic mucosa sample, a biopsy sample from the gastrointestinal tract, a colonic biopsy sample, a small intestine biopsy sample, a blood sample, a blood fraction, including peripheral blood mononuclear cells (PBMC) and peripheral blood lymphocytes (PBL). Preferably, the biological sample is a colonic mucosa sample or a blood sample.

The expressions “T regulatory lymphocytes with a CD4⁺ CD8αα^{low} Foxp3^{neg} phenotype”, “T regulatory lymphocytes characterized by a CD4⁺ CD8αα^{low} Foxp3^{neg} phenotype” and “DP8αTreg” are used interchangeably and refer to lymphocytes (primary culture cells or established cell lines derived from the primary culture cells) which express and display at their surfaces at least the cluster of differentiation molecules CD3, CD4, and the cluster of differentiation molecule CD8 consisting of the α/α homodimer, the level of expression of which being lower than that on T lymphocytes expressing the cluster of differentiation molecule CD8 consisting of the α/β heterodimer.

The expression “cell with a CD8αα^{low} phenotype” thus means that said cell expresses the homodimer α/α in a quantity lower than the quantity of chain a expressed by a T lymphocyte with a CD8α/β phenotype. Besides, a cell with a CD8αα^{low} phenotype does not express the heterodimer α/β.

DP8αTreg also express or over-express markers usually found on Foxp3⁺ Treg (e.g., CD25, CTLA4, GITR, LAG-3), and preferably express activation and co-stimulation markers (e.g., CD80, CD86, CD40L), adhesion markers (e.g., LFA-1, LFA3 and ICAM-1), IL-7R (CD127), particularly in the colonic lamina propria. Further, these T lymphocytes are characterized by a lack of expression of Foxp3 (called “Foxp3^{neg}” or “Foxp3⁻” T lymphocytes).

Upon activation, DP8αTreg secrete at least IFN-γ and interleukin-10 (IL-10), little if any IL-2. Preferably, these cells do not express IL-4, IL-5, IL-13, IL-17 and IL-22.

Preferably, the DP8αTreg are lamina propria lymphocytes (hereinafter abbreviated as “LPL”), peripheral blood lymphocytes (PBL), lymphocytes derived from naïve CD4 T cells, or established cell lines derived therefrom.

LPL are preferably obtained from a tissue sample of colonic mucosa.

LPL may be obtained by any suitable method well known by the skilled person.

A process for obtaining LPL may comprise the following steps:
separation of the lamina propria from the epithelium in a tissue sample of colonic mucosa, to obtain an isolated lamina propria,
digestion of the isolated lamina propria with collagenase and DNAse, to obtain a digested lamina propria,
filtration of the digested lamina propria, to obtain cells of the lamina propria free of mucus and large debris, and
centrifugation of said cells of the lamina propria, to obtain viable cells of the lamina propria, and
selection of the LPL, for example by selecting cells expressing the surface marker CD3 or by selective culture.

The separation of the Lamina propria (LP) from the epithelium in the tissue sample of colonic mucosa is for example carried out by incubating the tissue sample under agitation in EDTA buffer, for example for 30 minutes, followed by stripping and washing in a buffer, for example PBS. The lamina propria thereby isolated is then minced into 1 mm² fragments and washed with culture medium, for example RPMI containing penicillin (10%) and gentamycin (0.1 mg/ml; Sigma-Aldrich). Said tissue fragments are then digested with collagenase and DNAse (2 mg/ml each; Sigma-Aldrich), with shaking at 37° C. Mucus and large debris are removed by filtration through a 40-μm-cell strainer (BD). Viable cells are then obtained by Ficoll gradient centrifugation. LPL may then be selected by selecting cells expressing the surface marker CD3 or by a selective culture. An example of selective culture for obtaining lymphocytes is a culture wherein cells are stimulated with PHA (phytohemagglutinin) in the presence of irradiated feeder cells.

PBL may be obtained by any suitable method well-known by the skilled person. For example, PBL are obtained from a blood sample by elutriation.

PBL may also be isolated form PBMC.

PBMC (Peripheral Blood Mononuclear Cell) may be obtained by any suitable method well-known by the skilled person. For example, PBMCs are obtained from a blood sample, preferably a blood sample comprising an anticoagulant such as EDTA, by Ficoll gradient centrifugation.

In the context of the invention, the DP8αTreg are specific for F. prausnitzii (also called F. prausnitzii-induced DP8α Treg), i.e. they express T-cell receptors specific for F. prausnitzii so that they react specifically to antigen-presenting cells loaded with F. prausnitzii.

The term “antigen-presenting cell” (hereinafter abbreviated as “APC”) is intended to mean a cell that, after engulfing/internalizing and processing an antigen, displays the processed antigen complexed with major histocompatibility complexes (MHC) on their surfaces. Preferably, the APCs used in the present invention are professional APCs, more preferably Dendritic cells, Macrophages, Monocytes, gamma-delta T lymphocytes (Himoudi. J Immunol., 188(4):1708-16, 2012), as well as B-lymphocytes which express a B-cell receptor specific for an antigen of F. prausnitzii and which is able to further internalize and present this antigen at its surface associated with a class II MHC molecule. APCs useful in the present invention can also be non-professional APCs. Non-professional APCs do not constitutively express the major histocompatibility complexes, but stimulating with the appropriate cytokines triggers expression of class II MHC molecules. Non-professional APCs, together with the methods of stimulating them so that they express class II MHC molecules at their surfaces, are well known to the person of ordinary skill in the art, and are for instance described by Sundstrom J B and Ansari A A, Transpl Immunol, 4: 273-289, 1995. In the context of the invention, an “APC loaded with F. prausnitzii” and an “APC loaded with a fragment of F. prausnitzii” mean that an APC was incubated with F. prausnitzii or with a fragment of this bacteria, respectively, under culture condition and for a time sufficient to allow the F. prausnitzii, or the fragment where appropriate, to be internalized and processed by the APC, and then to allow the processed antigens from F. prausnitzii or from the fragment of this bacteria to be displayed associated with major histocompatibility complexes, for instance MHC class II (MHC II), on the surface of the APC.

The term “isolated” or “purified” with regard to a population of DP8αTreg as used herein refers to a cell population which either has no naturally-occurring counterpart or has been separated or purified from other components, including other cell types, which naturally accompany it, e.g., in normal or diseased tissues such as colon tissue, or body fluids such as blood. Typically, an isolated cell population is at least two-fold, four-fold, eight-fold, ten-fold, twenty-fold or more enriched for DP8αTreg when compared to the natural source from which the population was obtained. In an isolated population of DP8αT regulatory lymphocytes, the number of DP8αT regulatory lymphocytes represents at least 50%, 75%, 80%, 90%, 95% or, most preferably, 98% or 99% of the total cell number of the population

Isolating DP8αT regulatory lymphocytes (or a population of DP8αT regulatory lymphocytes) can be performed by using selective expression of surface markers unique to these cells. In particular, DP8αT regulatory lymphocytes may be sorted in a first time through positive selection of the cell surface proteins CD3, CD4, CD8 α/α, and in a second time through negative selection of cell surface protein Foxp3 (i.e. depletion of Foxp3⁺ cells). Optionally, the cell population is then further purified through positive selection of at least one, and by order of preference at least two, at least three, at least four, at least five, at least six, at least seven, at least eight, at least nine, at least ten, or all the cell surface protein(s) chosen from the group consisting of CD25, CTLA4, GITR, LAG-3, CD80, CD86, CD40L, LFA-1, LFA3 and ICAM-1 and CD127.

Methods for carrying out selection based on the presence or the absence of cell surface proteins are well-known to one skilled in the art. For instance, these cells may be isolated/purified by immunologic selection using antibodies which selectively bind to a selected cell surface protein.

The term “immunologic selection” refers to selecting, and optionally quantifying the number of, cells displaying specific cell surface proteins from a sample, or of cells comprising a cell surface protein of interest in a sample, by specific binding of the protein to an antibody or fragment thereof. Preferred immunologic selection methods useful in the methods of the present invention include cell staining, flow cytometry, fluorescence-activated cell sorting (FACS), magnetic bead purification (using magnetic beads coated with antibodies directed against a selected cell surface antigen), and the like.

The term “Inflammatory bowel disease” refers to a group of inflammation conditions characterized by a chronic and relapsing inflammation of the gastrointestinal tract as described by Baumgart and Sandbom (Lancet, 369: 1641-1657, 2007). In the context of the invention, the IBD is preferably a Crohn's disease or an ulcerative colitis. More preferably, the IBD is a Crohn's disease.

“Faecalibacterium prausnitzii” (abbreviated as “F. prausnitzii”) is a commensal bacterium of the human gut flora classified in the Firmicutes phylum, Clostridia class, Clostridiales order, Clostridiaceae family, and Faecalibacterium genus. This term refers to any strain of Faecalibacterium prausnitzii. By “a Faecalibacterium prausnitzii strain” is meant any bacterium which belongs to the Faecalibacterium prausnitzii species.

The term “isolated” or “purified” with regard to F. prausnitzii refers to a population of F. prausnitzii with a purity of at least 50%, by order of preference 75%, 80%, 90%, 95%, 98%, 99%, or more preferably 100%.

In the context of the invention, the term “immunogenic fragment” when referring to F. prausnitzii (e.g. live, live-attenuated or killed F. prausnitzii) is intended to mean any part of F. prausnitzii (e.g. cell wall or a component thereof such as peptidoglycan and/or proteins and/or peptides) once loaded by APC allows a processed antigen from the fragment to form a complex with major histocompatibility complexes, in particular CMHII, said complex being displayed at the surface of the APC so that it can activate/trigger expansion of DP8αTreg via TCR recognition.

In the context of the invention, the expression “proportion of” with regard to T regulatory lymphocytes with a CD4⁺ CD8αα^{low} Foxp3^{neg} phenotype is intended to mean the percentage of T regulatory lymphocytes with a CD4⁺ CD8αα^{low} Foxp3^{neg} phenotype relative to a whole population of given cells. For instance, the proportion of T regulatory lymphocytes with a CD4⁺ CD8αα^{low} Foxp3^{neg} phenotype to the CD4+ lymphocytes population is the percentage of T regulatory lymphocytes with a CD4⁺ CD8αα^{low} Foxp3^{neg} phenotype relative to the whole population of CD4+ lymphocytes.

As used herein, the terms “subject” and “patient” denote a human.

By “subject in need” is meant an individual suffering from or susceptible of suffering from IBD, an individual at risk for developing an IBD disease, or an individual that is in remission after having suffered from IBD.

The expression “healthy subject” refers to a subject who is not afflicted with IDB.

The term “treating” is meant to encompass both therapeutic and prophylactic methods, i.e. a method aiming at curing, improving the condition and/or extending the lifespan of an individual suffering from IBD. It also refers to methods aiming at preventing the appearance of IBD, as well as methods aiming at preventing a relapse.

As used herein, where applied to IBD, the term “preventing” is intended to mean that the onset of IBD is delayed or prevented.

By “effective amount” is meant an amount sufficient to achieve a concentration of compound which is capable of treating/preventing the disease to be treated/prevented. Such concentrations can be routinely determined by those of skilled in the art. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, etc.

As used herein, the term “at risk of developing an IBD” denotes a higher trend to suffer from an ID in the future.

### DETAILED DESCRIPTION OF THE INVENTION

### Methods of Diagnosing, Prognosing, and Predicting the Risk of Developing an Inflammatory Bowel Disease

### Method of Diagnosing an Inflammatory Bowel Disease

In a first aspect, the invention relates to an in vitro method of determining if a subject is afflicted with an inflammatory bowel disease comprising, or consisting of:

(a) determining the number and/or the concentration and/or proportion of T regulatory lymphocytes with a CD4⁺ CD8αα^{low} Foxp3^{neg} phenotype which are specific for F prausnitzii in a biological sample from the subject;

(b) optionally, comparing the result of step (a) with i) a control standard value corresponding to the number and/or concentration and/or proportion of these T regulatory lymphocytes typically found in a biological sample of the same nature from a healthy subject, and/or ii) a control standard value corresponding to the number and/or concentration and/or proportion of these T regulatory lymphocytes typically found in a biological sample of the same nature from a patient suffering from an inflammatory bowel disease; and

(c) deducting from the result(s) of step (a), and/or step (b) where appropriate, if the subject is afflicted with an inflammatory bowel disease.

In some embodiments, the method comprises a first step consisting of providing or obtaining a biological sample from the subject to be diagnosed.

Preferably, in this aspect of the invention, the subject to be diagnosed is suspected to be afflicted with an inflammatory bowel disease: thus the method can be performed to confirm that the subject is indeed suffering from an inflammatory bowel disease.

Preferably, the biological sample is a colonic mucosa sample, a blood sample or a blood fraction, including peripheral blood mononuclear cells (PBMC) and peripheral blood lymphocytes (PBL).

In a preferred embodiment, step (a) is carried out with a blood sample or a blood fraction. This embodiment is particularly advantageous because collecting a blood sample is both easier and less invasive than performing colonoscopy and colonic biopsy, the standard tests currently requested for diagnosing IBD.

The “control standard value” used in step (b) may be obtained by, for example, determining the proportion and/or number and/or concentration of T regulatory lymphocytes with a CD4⁺ CD8αα^{low} Foxp3^{neg} phenotype which are specific for F. prausnitzii in a biological sample (e.g. colonic mucosa, blood . . . ) of a given population of subjects (e.g healthy subjects, patients suffering from IBD, patients in remission) and obtaining an average or median figure.

As will be clear to the skilled person, the nature of the comparison of the proportion, number and/or concentration of CD4⁺ CD8αα^{low} Foxp3^{neg} phenotype specific for F. prausnitzii determined in a subject biological sample to be tested, with the control and the conclusion drawn in step (c) will depend on the nature of the control.

A proportion, number or concentration of DP8αTreg specific for F. prausnitzii in the biological sample of the subject to be tested equal to, similar to or greater than, the corresponding healthy subject control standard value may be indicative that the patient is not afflicted with an IBD.

In contrast, a proportion, number or concentration value of DP8αTreg specific for F. prausnitzii lower than the corresponding healthy subject control standard value may be indicative that the patient is afflicted with an IBD.

Similarly, a proportion, number or concentration of DP8αTreg specific for F. prausnitzii equal to, similar to or lower than, the corresponding IBD patient control standard value may be indicative that the patient suffers from an IBD.

### Method of Prognosing Patients Afflicted with an Inflammatory Bowel Disease

A second aspect of the invention relates to an in vitro method of prognosing outcome of an inflammatory bowel disease in a patient, the method comprises, or consists of, a step of determining the number and/or concentration and/or proportion of T regulatory lymphocytes with a CD4⁺ CD8αα^{low} Foxp3^{neg} phenotype which are specific for F prausnitzii in a biological sample from the patient, the greater the proportion of T regulatory lymphocytes with a CD4CD8αα^{low} phenotype which are specific for Faecalibacterium prausnitzii, the better the prognosis.

An embodiment of this aspect of the invention comprises, or consists of, the following steps:

(a) determining the number and/or the concentration and/or proportion of T regulatory lymphocytes with a CD4⁺ CD8αα^{low} Foxp3^{neg} phenotype which are specific for F prausnitzii in a biological sample from the patient; and

(b) deducing from the result of step a) the prognosis of said patient.

In some embodiments, the method comprises a first step consisting of providing or obtaining a biological sample from the patient afflicted with an inflammatory bowel disease.

In a preferred embodiment, step (a) is carried out with a blood sample or a blood fraction.

In a patient afflicted with an inflammatory bowel disease, a low level of T regulatory lymphocytes with a CD4⁺ CD8αα^{low} Foxp3^{neg} phenotype specific for F prausnitzii is likely indicative of a poor prognosis. On the contrary, the higher the number of these specific Treg is, the better the prognosis is.

Hence, the measurement of no or a low number and/or concentration of T regulatory lymphocytes with a CD4⁺ CD8αα^{low} Foxp3^{neg} phenotype which are specific for F prausnitzii in step a) is indicative of a poor prognosis, whereas the measurement of high levels of T regulatory lymphocytes with a CD4⁺ CD8αα^{low} Foxp3^{neg} phenotype which are specific for F prausnitzii in step b) is indicative of a good prognosis.

As used throughout the present specification, the term “poor prognosis” refers to a patient who is likely to experience an early relapse and/or to not respond to a new treatment recently initiated. The term “good prognosis” refers to a patient who is likely to have long periods of remission and/or to have a good response to a new treatment recently initiated.

As indicated above, the number and/or concentration and/or proportion of T regulatory lymphocytes with a CD4⁺ CD8αα^{low} Foxp3^{neg} phenotype specific for F prausnitzii determined in step a) provides an evaluation of the prognosis of the outcome of an inflammatory bowel disease in a patient afflicted with this condition. Therefore, in a preferred embodiment of the in vitro method of the invention, in order to evaluate the prognosis, the value obtained in step a) is applied to a standard calibration curve showing a relationship between the number and/or concentration and/or proportion of T regulatory lymphocytes with a CD4⁺ CD8αα^{low} Foxp3^{neg} phenotype specific for F prausnitzii in the biological sample and the probable outcome (relapse, short or long period of remission, progression to a severe form of the disease . . . ).

The standard calibration curve can be obtained by determining the number and/or concentration and/or proportion of T regulatory lymphocytes with a CD4⁺ CD8αα^{low} Foxp3^{neg} phenotype specific for F prausnitzii in a large cohort of patients whose outcome is known.

In one embodiment, the biological sample is obtained prior to the patient receiving any therapy.

In another embodiment, the biological sample is obtained after initiation of a treatment of IBD, in particular after initiation of a treatment according to the invention as disclosed below.

### Method of Predicting the Risk of Developing an Inflammatory Bowel Disease

The inventors have shown that the level of T regulatory lymphocytes with a CD4⁺ CD8αα^{low} Foxp3^{neg} phenotype which are specific for F prausnitzii in a patient, in particular in patient's blood and patient's colonic mucosa, is associated with the intestinal immunologic homeostasis and with the development of inflammatory bowel diseases.

Therefore, a third aspect of the invention relates to an in vitro method of predicting whether a subject is at risk of developing an inflammatory bowel disease, said method comprising, or consisting of:

a) determining the number and/or concentration and/or proportion of T regulatory lymphocytes with a CD4⁺ CD8αα^{low} Foxp3^{neg} phenotype which are specific for F prausnitzii in a biological sample from the subject;

b) predicting from the result of step (a) if the subject is at risk of suffering from an inflammatory bowel disease, wherein a low value at step (a), relative to a reference value for a sample of the same nature, indicates that said subject is at risk of developing an inflammatory bowel disease.

In some embodiments, the method comprises a first step consisting of providing or obtaining a biological sample from the subject to be tested.

In a preferred embodiment, step (a) is carried out with a blood sample or a blood fraction.

In the third aspect of the invention, the subject to be tested is not yet afflicted with an inflammatory bowel disease.

In an embodiment, the reference value (number and/or concentration and/or proportion of T regulatory lymphocytes with a CD4⁺ CD8αα^{low} Foxp3^{neg} phenotype specific for F. prausnitzii) is the value determined in a biological sample from a subject who is not afflicted with an inflammatory bowel disease, said biological sample is of the same nature than that of the subject to be tested. The lower the value determined in step (a), the higher the risk of developing an inflammatory bowel disease in the future.

Determining the Number and/or Concentration and/or Proportion of T Regulatory Lymphocytes Specific for F prausnitzii with a CD4⁺ CD8αα^{low} Foxp3^{neg} Phenotype

The step of determining the number and/or concentration and/or proportion of T regulatory lymphocytes specific for F prausnitzii with a CD4⁺ CD8αα^{low} Foxp3^{neg} phenotype in the first to the third aspects of the invention is typically carried out in two steps:
a step (I) of immunologic selection using i) positive selection based on the presence of the surface proteins CD3, CD4, CD8α, and optionally on the presence of all or part of the surface proteins CD25, CTLA4, GITR, LAG-3, CD80, CD86, CD40L, LFA-1, LFA3 and ICAM-1 and CD127, combined with ii) negative selection against cells comprising the surface markers specific to non-DP8αTreg, such as FOXP3 and CD8β.

Methods of immunosorting using labelled-antibodies specific to the proteins displayed at the surface of DP8αTreg are particularly suitable for quantifying DP8αTreg. These methods are known in the art. Preferred immunologic selection/immunosorting methods useful in the method of the invention include flow cytometry, in particular Fluorescence-activated cell sorting (FACS).
a step (II) of determining the number of the DP8αTreg obtained in step (I) which are specific to F. prausnitzii. To do so, the DP8αTreg obtained in step (I) can be brought into contact with APCs loaded with F. prausnitzii or fragments of F. prausnitzii under conditions which allow cells specific to F. prausnitzii to be activated. For instance, the percentage of F. prausnitzii-induced DP8α Treg can be determined by measuring (for example by flow cytometry) the number of the DP8αTreg obtained in step (I) which secrete cytokines typically secreted by regulatory T cells, in particular IFN-γ and/or IL-10, after being contacted with APCs loaded with F. prausnitzii or fragments of F. prausnitzii. The APCs used to perform step (II) can be professional or non-professional APCs. Preferably, the APCs are professional APCs, they are more preferably chosen from the group consisting of Dendritic cells, Macrophages or Monocytes.

In an embodiment, step (I) is carried out before step (II). Alternatively, step (II) can be carried out before step (I).

The protocols provided in the examples of the present application may for instance be used.

Methods of Treating or Preventing an Inflammatory Bowel Disease

Adoptive Immunotherapy

In a fourth aspect, the invention relates to a method of treating or preventing an inflammatory bowel disease in a subject in need of such therapy, the method is an adoptive immunotherapy comprising, or consisting of, a step of administering to the patient a therapeutically effective amount of a population of isolated T regulatory lymphocytes with a CD4⁺ CD8αα^{low} Foxp3^{neg} phenotype which are specific for F. prausnitzii.

A fifth aspect of the invention relates to T regulatory lymphocytes, preferably a population of isolated regulatory lymphocytes, with a CD4⁺ CD8αα^{low} Foxp3^{neg} phenotype which are specific for F. prausnitzii, for use as a medicament in the treatment of an inflammatory bowel disease.

Preferably, the isolated T regulatory lymphocytes with a CD4⁺ CD8αα^{low} Foxp3^{neg} phenotype which are specific for F. prausnitzii used to carry out these aspects of the invention are isolated from lamina propria (i.e. lamina propria lymphocytes) or from blood (i.e. peripheral blood lymphocytes).

In a preferred embodiment, the isolated population of T regulatory lymphocytes with a CD4⁺ CD8αα^{low} Foxp3^{neg} phenotype specific for F. prausnitzii are obtained from the subject to be treated (i.e. autologous lymphocytes). Use of autologous lymphocytes is particularly advantageous since rejection of the administered lymphocytes by the recipient is avoided.

In an alternative preferred embodiment, the isolated population of T regulatory lymphocytes with a CD4⁺ CD8αα^{low} Foxp3^{neg} phenotype specific for F. prausnitzii are allogeneic lymphocytes, i.e. the Treg are obtained from a human donor who is not the subject to be treated.

Rejection of the allogeneic lymphocytes by the subject to be treated may be prevented by the administration to the patient of immunosuppressive drugs prior and/or after administration of the isolated population of T regulatory lymphocytes characterised a CD4⁺ CD8αα^{low} Foxp3^{neg} phenotype specific for F. prausnitzii. Preferably, the human donor is a healthy donor.

The main advantage of this alternative embodiment is that allogeneic lymphocyte populations can be chosen within a lymphobank which is immediately available.

The antigens on the surfaces of transplanted tissue that most strongly provoke rejection are the blood group (ABO) antigens and the major histocompatibility complex (MHC) proteins (in the human also called leukocyte antigen (HLA) proteins). Therefore, to limit the risk of rejection, the allogeneic lymphocyte population which best matches with the patient to be administered for these antigens will be chosen.

The HLA proteins, encoded by clusters of genes that form a region located on chromosome 6 known as the Major Histocompatibility Complex (or MHC), play an important role in graft rejection.

The MHC genes are polygenic, i.e. each individual possesses multiple, different MHC class I and MHC class II genes.

The MHC genes are also polymorphic, i.e. many variants of each gene are present in the human population. Each MHC Class I receptor consists of a variable alpha chain and a relatively conserved beta2-microglobulin chain. Three different, highly polymorphic class I alpha chain genes have been identified. These are called HLA-A, HLA-B, and HLA-C. In humans, there are three pairs of MHC class II [alpha] and [beta] chain genes, called HLA-DR, HLA-DP, and HLA-DQ.

Thus, to determine patient/donor matching, the Class I HLA-A, Class I HLA-B, Class I HLA-C, Class II HLA-DP, Class II HLA-DQ Class II HLA-DR, and/or blood “ABO” antigen types of both patient and donor have to be compared.

Typing of MHC (HLA) antigens and the blood group (ABO) antigens can be performed by various means very well known to a person of ordinary skill in the art. For example, typing may be performed (i) by serology, using antibodies specific for particular MHC molecules to detect the presence of the targeted MHC molecules on donor or recipient cells, e.g., by the lymphocytotoxicity test; or (ii) by direct analysis of the nucleotide sequence of the DNA of the MHC alleles (for instance by using methods which employ sequence-specific oligonucleotide primers and amplification by polymerase chain reaction (PCR), and/or fluorescent detection methods which use a DNA chip to which are bound sequence specific oligonucleotides designed to detect unique sequences present in the different MHC alleles).

Isolated T Regulatory Lymphocytes with a CD4⁺ CD8αα^{low} Foxp3^{neg} Phenotype Specific for F. prausnitzii

The population of isolated T regulatory lymphocytes with a CD4⁺ CD8αα^{low} Foxp3^{neg} phenotype specific for F. prausnitzii used in the fourth and five aspects of the invention, whatever the embodiment, are primary culture cells or established cell lines derived from the primary culture cells.

The isolated T regulatory lymphocytes with a CD4⁺ CD8αα^{low} Foxp3^{neg} phenotype which are specific for F. prausnitzii may be isolated from a biological sample. Said biological sample may be as defined above in the section “Definitions”.

In a preferred embodiment, the isolated T regulatory lymphocytes with a CD4⁺ CD8αα^{low} Foxp3^{neg} phenotype which are specific for F. prausnitzii are isolated from a blood sample or a colonic mucosa sample.

Isolating T regulatory lymphocytes with a CD4⁺ CD8αα^{low} Foxp3^{neg} phenotype specific for F. prausnitzii can be carried out by using positive and negative selection according to the surface proteins which are, or which are not, expressed by the T regulatory lymphocytes with a CD4⁺ CD8αα^{low} Foxp3^{neg} phenotype. Methods of isolating/selecting cells based on the presence or the absence of cell surface proteins are well-known to one skilled in the art. For instance, these cells may be isolated/purified by immunologic selection/immunosorting using antibodies which selectively bind to a selected cell surface protein. Isolation/purification of T regulatory lymphocytes with a CD4⁺ CD8αα^{low} Foxp3^{neg} phenotype may be carried out as described in the paragraph entitled “Cell isolation and cell line generation” of Example 1 of the invention.

The isolated T regulatory lymphocytes with a CD4⁺ CD8αα^{low} Foxp3^{neg} phenotype which are specific for F. prausnitzii may also be obtained from naïve CD4 T cells according to the following process.

The present invention also relates to a process, in particular an in vitro process, for obtaining T regulatory lymphocytes with a CD4⁺ CD8αα^{low} Foxp3^{neg} phenotype which are specific for F. prausnitzii, said process comprising the following steps:
a) culturing APCs (Antigen-presenting Cells) in the presence of F. prausnitzii and, optionally, in the presence of butyrate, thereby obtaining F. prausnitzii loaded APCs, and
b) culturing naïve CD4 T cells in the presence of:
   the F. prausnitzii loaded APCs obtained in step a),
   optionally IL-2, and
   optionally butyrate,
thereby obtaining T regulatory lymphocytes with a CD4⁺ CD8αα^{low} Foxp3^{neg} phenotype which are specific for F. prausnitzii.

The aim of step a) is to obtain F. prausnitzii loaded APCs.

APCs may be as defined above in the section “definitions”.

In a preferred embodiment, the APCs used in step a) are monocytes and/or monocyte-derived immature dendritic cells.

Monocytes may for example be obtained from PBMC by any method well known by the skilled person. For example, monocytes are isolated from PBMC using the CD14 microbeads positive selection kit of Miltenyi.

Monocyte-derived immature dendritic cells may be obtained from monocytes by any method well known by the skilled person. For example, monocyte-derived immature dendritic cells are obtained by culturing monocytes (CD14+) in the presence of IL-4 and GM-CSF. The culture of the monocytes in the presence of IL-4 and GM-CSF may be of at least 2 days, preferably at least 3 days, for example 3 days. The concentration of IL-4 may be of 200 IU/ml to 500 IU/ml, preferably 200 IU/ml to 300 IU/ml, for example 200 IU/ml. The concentration of GM-CSF may be of 100 IU/ml to 1000 IU/ml, preferably 500 IU/ml to 1000 IU/ml, for example 1000 IU/ml.

The culture of step a) may be carried out for 10 hours to 16 hours, preferably for 12 hours to 14 hours.

In step a), the monocytes and/or monocyte-derived immature dendritic cells may be cultured in any suitable culture medium. Culture media suitable for the culture of monocytes and/or monocyte-derived immature dendritic cells are well known by the skilled person.

For example, the culture step a) may be carried out in a medium comprising or consisting of RPMI, preferably supplemented with Fetal calf serum, human serum albumin or human plasma.

The aim of step b) is to induce the differentiation of naïve CD4 T cells into T regulatory lymphocytes with a CD4⁺ CD8αα^{low} Foxp3^{neg} phenotype which are specific for F. prausnitzii.

Naïve CD4 T cells are characterized by the phenotype CD4 CD45 RA+RO−.

Naïve CD4 T cells may be obtained from PBMC or PBL by any method well known by the skilled person. For example, naïve CD4 T cells are obtained from PBMC or PBL using the EasySep negative selection kit for naive CD4 T cells (STEMCELL technologies).

The culture of step b) may be carried out for at least 2 days, preferably at least 3 days, for example from 4 to 12 days, preferably for 4 to 10 days, more preferably for 4 to 6 days.

The culture of step b) is for example carried out during 4, 5 or 6 days.

The culture of step b) may be performed in any suitable culture medium. Culture media suitable for the culture of naïve CD4 T cells, monocytes and/or monocyte-derived immature dendritic cells are well known by the skilled person.

For example, the culture step b) may be carried out in a medium comprising or consisting of RPMI, preferably supplemented with a pool of human serum.

IL-2 may be used in step b) to improve the efficiency of the differentiation or proliferation of the naïve CD4 T cells. IL-2 is for example added in the culture medium of step b).

Butyrate may be used in step a) and/or in step b) to improve the efficiency of the differentiation of the naïve CD4 T cells.

Butyrate is for example added in the culture medium of step a) and/or in the culture medium of step b).

The concentration of butyrate in the culture medium is preferably comprised from 0.125 mM to 0.5 mM, more preferably from 0.125 mM to 0.25 mM. For example, a preferred concentration of butyrate in the culture medium is 0.145 mM.

Butyrate (also called “butanoate”) is the conjugate base of butyric acid (also known as butanoic acid). The formula of butyrate is C₄H₇O₂⁻.

A process for obtaining T regulatory lymphocytes with a CD4⁺ CD8αα^{low} Foxp3^{neg} phenotype which are specific for F. prausnitzii is for example disclosed in Example 8.

Primary culture cells are cells isolated directly from living tissue (for example a tissue sample or a blood sample). These cells have undergone very few population doublings following their isolation.

Methods of generating cell lines derived from primary culture cells are known to the person having ordinary skill in the art, and includes in particular the methods described by Gregori et al. (Methods Mol Biol., 677: 31-46, 2011). For instance, T cell lines can be generated by stimulations with PHA, irradiated feeder cells and IL-2, as described by Fonteneau et al. (J Immunol 159, 2831-2839, 1997).

Preferably, the population of isolated T regulatory lymphocytes with a CD4⁺ CD8αα^{low} Foxp3^{neg} phenotype which are specific for F. prausnitzii used to carry out the fourth and fifth aspects of the invention, whatever the embodiment, were previously activated and expanded.

Activation and/or expansion (also called “proliferation”) of the T regulatory lymphocytes with a CD4⁺ CD8αα^{low} Foxp3^{neg} phenotype specific for F. prausnitzii can be performed by contacting these Treg with APCs loaded with F. prausnitzii or fragments of F. prausnitzii under conditions which allow cells specific to F. prausnitzii to be activated. Activation may be assessed by determining if the isolated T regulatory lymphocytes with a CD4⁺ CD8αα^{low} Foxp3^{neg} phenotype (cells line or primary cells) express cytokines typically secreted by regulatory T cells, in particular TNF-α, IFN-γ and/or IL-10.

The APCs can be professional or non-professional APCs. Preferably, the APCs are professional APCs. More preferably, APCs are chosen from the group consisting of Dendritic cells, Macrophages or Monocytes.

Proliferation of T regulatory lymphocytes with a CD4⁺ CD8αα^{low} Foxp3^{neg} phenotype can also be triggered ex vivo (or in vitro) upon CD3 activation and/or CD28 activation (e.g. proliferation can be induced by using an antibody directed against these surface proteins).

A preferred embodiment of the method according to the fourth aspect of the invention comprises, or consists of, the following steps:

(i) isolating a population of T regulatory lymphocytes with a CD4⁺ CD8αα^{low} Foxp3^{neg} phenotype which are specific for F. prausnitzii;

(i′) optionally, generating a cell line from isolated population of T regulatory lymphocytes with a CD4⁺ CD8αα^{low} Foxp3^{neg} phenotype which are specific for F. prausnitzii of step (i);

(ii) activating and/or expanding the isolated population of T regulatory lymphocytes with a CD4⁺ CD8αα^{low} Foxp3^{neg} phenotype which are specific for F. prausnitzii of step (i), or the cells lines of step (i′) where appropriate, by culturing these cells in a culture medium comprising:
a compound suitable for activating CD3 and/or CD28 pathway(s) (e.g. anti-CD3 anti-CD28 antibodies, phorbolmyristate acetate and/or calcium ionophore); and/or
APCs loaded with F. prausnitzii or fragments of F. prausnitzii;

(iii) administering the activated and/or expanded T regulatory lymphocytes with a CD4⁺ CD8αα^{low} Foxp3^{neg} phenotype which are specific for F. prausnitzii of step (ii) to the patient in a therapeutically effective amount.

Preferably, the population of T regulatory lymphocytes with a CD4⁺ CD8αα^{low} Foxp3^{neg} phenotype specific for F. prausnitzii is isolated from the subject to be treated.

In the context of the invention, the T regulatory lymphocytes with a CD4⁺ CD8αα^{low} Foxp3^{neg} phenotype specific for F. prausnitzii used to carry out the fourth and fifth aspects of the invention, whatever the embodiment, are preferably formulated into a pharmaceutical composition in an effective amount (i.e. an amount which is capable of treating/preventing the IBD without causing overly negative effects in the administered subject).

The dosage of T regulatory lymphocytes with a CD4⁺ CD8αα^{low} Foxp3^{neg} phenotype specific for F. prausnitzii required to in the subject to be treated can vary according to numerous factors, the presence and the nature of co-stimulatory molecules (e.g. cytokines . . . ), the mode of administration, the age, weight, condition of the subject, the route of administration, the frequency of administration, the other ingredients in the pharmaceutical composition, and the severity of the disease. Generally, T regulatory lymphocytes with a CD4⁺ CD8αα^{low} Foxp3^{neg} phenotype specific for F. prausnitzii for use in the treatment or prevention an IBD may be administered in the range from about 10⁵ cells/kg to about 10⁸ cells/kg, alternatively from about 10⁷ cells/kg to about 10⁸ cells/kg, and preferably from about 0.1×10⁶ cells/kg to 5×10⁶ cells/kg.

Examples of suitable routes of administration include, but not limited to, parenteral routes, including for instance intramuscular, subcutaneous, intravenous, transarterial, intraperitoneal, to a mucosal surface (e.g. rectal, colon, small intestine, gastrointestinal tract . . . ). In some embodiments, the administration can be given at multiple locations.

Preferably, the T regulatory lymphocytes with a CD4⁺ CD8αα^{low} Foxp3^{neg} phenotype specific for F. prausnitzii, or the pharmaceutical composition comprising these Treg, is administered intraperitoneally and/or in the gastrointestinal tract and/or to a mucosal surface (preferably the colonic mucosa).

Active Immunotherapy

In a sixth aspect, the invention relates to a method of treating or preventing an inflammatory bowel disease in a subject in need of such therapy, the method comprising, or consisting of, a step of in vivo activating and/or inducing the proliferation of T regulatory lymphocytes with a CD4⁺ CD8αα^{low} Foxp3^{neg} phenotype specific for F. prausnitzii by administering a therapeutically effective amount of at least one compound chosen from the group consisting of:

(i) an isolated F. prausnitzii strain;

(ii) an isolated live-attenuated F. prausnitzii strain;

(iii) an isolated killed F. prausnitzii strain;

(iv) at least one fragment of at least one of strains (i), (ii) and (iii);

(v) antigen-presenting cells (APCs), preferably isolated APCs, loaded with at least one of the compounds (i), (ii), (iii) and (iv).

In a seventh aspect, the invention relates a composition comprising:

(i) a F. prausnitzii strain; and/or

(ii) a live-attenuated F. prausnitzii strain; and/or

(iii) a killed F. prausnitzii strain; and/or

(iv) at least one fragment of at least one of strains (i), (ii) and (iii); and/or

(v) antigen-presenting cells (APCs), preferably isolated APCs, loaded with at least one of (i), (ii), (iii) and (iv);

as a medicament for activating and/or inducing the proliferation of T regulatory lymphocytes with a CD4⁺ CD8αα^{low} Foxp3^{neg} phenotype which are specific for F. prausnitzii in the treatment of an inflammatory bowel disease.

The F. prausnitzii strain (i), (ii), (iii), the fragment (iv) and the loaded APC (v) described herein are preferably formulated into a pharmaceutical composition.

Protocols for loading APC with (i), (ii), (iii) or (iv) are well known to the skilled artisan. The protocols provided in the examples of the present application may for instance be used.

The APCs of (v) can be obtained either from the subject to be treated (autologous APCs) or from a donor (allogeneic APCs). Preferably, the APCs are obtained from the subject to be treated.

When the APCs of (v) are obtained from a donor, to limit the risk of rejection of the administered APCs, preferably the allogeneic APCs are chosen so that they best matches with the subject to be treated. As described above, to determine patient/donor matching, the Class I HLA-A, Class I HLA-B, Class I HLA-C, Class II HLA-DP, Class II HLA-DQ Class II HLA-DR, and/or blood “ABO” antigen types of both subject and donor have to be compared.

Preferably, the APCs are professional APCs. More preferably, APCs are chosen from the group consisting of Dendritic cells, Macrophages, Monocytes or B-lymphocytes which express a B-cell receptor specific for an antigen of F. prausnitzii and which is able to further internalize and present this antigen at its surface associated with a class II MHC molecule.

The F. prausnitzii strain (i), (ii), (iii), the fragment (iv) and the loaded APC (v) are used in the pharmaceutical composition in an effective amount, i.e. an amount which is able to induce activation and/or expansion of T regulatory lymphocytes with a CD4⁺ CD8αα^{low} Foxp3^{neg} phenotype which are specific for F. prausnitzii, so that the administered Treg are capable of treating/preventing the IBD without causing overly negative effects in the administered subject.

The dosage of the F. prausnitzii strain (i), (ii), (iii), the fragment (iv) and the loaded APC (v) required to induce activation and/or expansion of T regulatory lymphocytes with a CD4⁺ CD8αα^{low} Foxp3^{neg} phenotype specific for F. prausnitzii in the subject to be treated can vary according to numerous factors, including the specific antigen (i.e. compounds (i), (ii), (iii), (iv) or (v)) or combination thereof being utilized, the presence and the nature of co-stimulatory molecules (e.g. cytokines . . . ), the mode of administration, the age, weight, condition of the subject, the route of administration, the frequency of administration, the other ingredients in the pharmaceutical composition, and the severity of the disease.

Generally, the loaded APCs for use in the treatment or prevention of an IBD according to the sixth and seventh aspects of the invention may be administered in the range from about 10³ cells/kg to about 10⁷ cells/kg, and preferably from about 10⁵ cells/kg to 10⁶ cells/kg. A F. prausnitzii strain may be administered in the range from about 10⁴ bacteria/kg to about 10⁸ bacteria/kg, alternatively from about 10⁶ bacteria/kg to about 10⁷ bacteria/kg, preferably from about 10⁵ bacteria/kg to about 10⁶ bacteria/kg. A live-attenuated F. prausnitzii strain may be administered in the range from about 10⁴ bacteria/kg to about 10⁸ bacteria/kg, alternatively from about 10⁶ bacteria/kg to about 10⁷ bacteria/kg, preferably from about 10⁵ bacteria/kg to about 10⁶ bacteria/kg. A killed F. prausnitzii strain may be administered in the range from about 10⁴ bacteria/kg to about 10⁸ bacteria/kg, alternatively from about 10⁶ bacteria/kg to about 10⁷ bacteria/kg, preferably from about 10⁵ bacteria/kg to about 10⁶ bacteria/kg.

Examples of suitable routes of administration include, but are not limited to, parenteral routes, including for instance intramuscular, subcutaneous, intravenous, transarterial, intraperitoneal, to a mucosal surface (e.g. rectal, colon, small intestine, gastrointestinal tract . . . ). In some embodiments, the administration can be given at multiple locations.

The compounds (i) to (v), in particular the loaded APCs, can also be administered directly to an appropriate lymphoid tissue, in particular the mucosal-associated lymphoid tissue.

In an advantageous embodiment, the compounds (i) to (v), or the pharmaceutical composition which comprises one or more of these compounds, is administered intraperitoneally and/or to a mucosal surface (e.g. rectal, colon, small intestine, gastrointestinal tract . . . ) and/or in the mucosal-associated lymphoid tissue of the gut and/or in the gut-associated lymphoid tissues (i.e. Peyer's patches, lymphoid follicles and/or lamina propria).

As used herein, the term “pharmaceutical composition” refers to a carrier medium or or diluent which is not prejudicial, and which is not excessively toxic, to the subject to be treated at the concentration at which it is administered.

Further, the pharmaceutical composition does not interfere with the effectiveness of the biological activity of, and is not detrimental to, the active substances (i.e. in the context of the fourth and fifth aspects of the invention, the DP8αTreg specific for F. prausnitzii strain; in the context of the fifth and seventh aspects of the invention the F. prausnitzii strain (i), (ii), (iii), the fragment (iv) and the loaded APC (v)).

Pharmaceutically acceptable carriers can be routinely selected by those skilled in the art in accordance with the mode of administration and the nature of the active substances (i.e. in the context of the fourth and fifth aspects of the invention, the DP8αTreg specific for F. prausnitzii strain; in the context of the fifth and seventh aspects of the invention the F. prausnitzii strain (i), (ii), (iii), the fragment (iv) and the loaded APC (v)). Examples of suitable pharmaceutical compositions include, but are not limited to, physiological water (NaCl 0.9%), salt solution (e.g., Ringer's solution), human serum albumin solution . . . .

When the active substances are living cells, the pharmaceutical composition must be carefully chosen not to be toxic for them.

Method of Monitoring the Efficacy of a Treatment of an Inflammatory Bowel Disease

In an eighth aspect, the invention further provides a method of monitoring the efficacy of a preventive or curative treatment of an inflammatory bowel disease, comprising monitoring the number and/or concentration and/or proportion of T regulatory lymphocytes with a CD4⁺ CD8αα^{low} Foxp3^{neg} phenotype which are specific for F prausnitzii in a biological sample from the subject during the treatment.

An increase in the number and/or concentration and/or proportion of T regulatory lymphocytes with a CD4⁺ CD8αα^{low} Foxp3^{neg} phenotype which are specific for F prausnitzii over the course of the treatment may indicate that the treatment is effective.

Preferably, the treatment comprises, or consists in administering immunosuppressant (notably azathioprine, 6-mercaptopurine, methotrexate, tacrolimus, ciclosporin), biologics (notably monoclonal antibodies including anti-TNFalpha), probiotics, (i.e. live microorganisms that may confer a health benefit on the host) or antibiotics

Alternatively, the treatment comprises, or consists in, administering F prausnitzii (a live, live-attenuated; and/or killed F. prausnitzii strain). Preferably, the probiotics and the F prausnitzii are formulated into a pharmaceutical composition as described above.

Preferably, the biological sample is a colonic mucosa sample, a blood sample or a blood fraction, including peripheral blood mononuclear cells (PBMC) and peripheral blood lymphocytes (PBL).

Kits According to the Invention

The invention provides kits that are useful in the above methods and uses of the invention.

A ninth aspect of the invention relates to a kit for diagnosing, prognosing and/or predicting the risk of developing an IBD, and/or for monitoring the efficacy of a treatment of an IBD Such a kit comprises means for determining the number and/or concentration and/or proportion of T regulatory lymphocytes with a CD4⁺ CD8αα^{low} Foxp3^{neg} phenotype specific for F prausnitzii. Such means include antibodies specific for the surface proteins specific to DP8αTreg (e.g. CD3, CD4, CD8α, CD25, CTLA4, GITR, LAG-3, CD80, CD86, CD40L, LFA-1, LFA3 and ICAM-1 and CD127) to allow for a positive selection based on the presence of these proteins, and antibodies specific for surface markers specific to non-DP8αTreg, such as FOXP3 and CD8β, to carry out a negative selection.

The antibodies used in the kit can be labeled with detectable compound such as fluorophores or radioactive compounds. Alternatively, the kit may further comprise a secondary antibody, labeled with a detectable compound, which binds to an unlabelled primary antibody.

According to an embodiment, the kit comprises, in addition to the means for determining the number and/or concentration and/or proportion of T regulatory lymphocytes with a CD4⁺ CD8αα^{low} Foxp3^{neg} phenotype specific for F prausnitzii, one or more control samples (“control standard value”) comprising a known number and/or concentration and/or proportion of T regulatory lymphocytes with a CD4⁺ CD8αα^{low} Foxp3^{neg} phenotype specific for F prausnitzii, said number and/or concentration and/or proportion being representative of a given population of subjects (e.g healthy subjects, patients suffering from an IBD, patients in remission, patient at risk of developing the disease).

Further, the kit can comprise instructions for the use of said kit i) in predicting whether a subject is at risk of developing an IBD, and/or ii) in diagnosing an IBD, and/or iii) in prognosing an IBD.

The kit can also comprise a standard calibration curve showing a relationship between the number and/or concentration and/or proportion of T regulatory lymphocytes with a CD4⁺ CD8αα^{low} Foxp3^{neg} phenotype specific for F prausnitzii in the biological sample and the probable outcome of the disease (relapse, short or long period of remission, progression to a severe form of the disease . . . ).

The standard calibration curve can be obtained by determining the number and/or the concentration and/or proportion of T regulatory lymphocytes with a CD4⁺ CD8αα^{low} Foxp3^{neg} phenotype specific for F prausnitzii in a large cohort of patients whose outcome is known.

A tenth aspect of the invention further relates to a kit for treating or preventing an inflammatory bowel disease comprising, or consisting of:
a packaging material;
a known amount of:
   (i) a population of isolated regulatory lymphocytes, with a CD4⁺ CD8αα^{low} Foxp3^{neg} phenotype, which are specific for F. prausnitzii, or a pharmaceutical composition thereof; and/or
   (ii) an isolated F. prausnitzii strain, or a pharmaceutical composition thereof; and/or
   (iii) an isolated live-attenuated F. prausnitzii strain, or a pharmaceutical composition thereof; and/or
   (iiii) an isolated killed F. prausnitzii strain, or a pharmaceutical composition thereof; and/or
   (v) at least one fragment of at least one of strains (ii), (iii) and (iv), or a pharmaceutical composition thereof; and/or
   (vi) antigen-presenting cells (APCs), preferably isolated APCs, loaded with at least one of (i), (ii), (iii) and (iv); loaded with at least one of the compounds (ii), (iii), (iv) and (v), or a pharmaceutical composition thereof, and/or
a label or package insert contained with said packaging material indicating that the “drugs” (i) to (vi), or pharmaceutical compositions thereof, are effective in the prevention and/or treatment of an IBD, preferably the Crohn's disease, in a subject suffering from an IBD or at risk of developing an IBD.

In a particular embodiment, the article of manufacture as described herein comprises a label or package insert further indicating the list of contraindications for the treatment with at least one drug chosen from the group consisting of the drugs (i) to (vi).

Preferably, the population of isolated regulatory lymphocytes, with a CD4⁺ CD8αα^{low} Foxp3^{neg} phenotype, which are specific for F. prausnitzii is an activated population.

In a preferred implementation of the first to the tenth aspects of the invention according to any one of the embodiments, the inflammatory bowel disease is the Crohn's disease.

All references cited herein, including journal articles or abstracts, published patent applications, issued patents or any other references, are entirely incorporated by reference herein, including all data, tables, figures and text presented in the cited references.

The invention will be further illustrated by the following figure and examples. However, these examples and figure should not be interpreted in any way as limiting the scope of the present invention.

### BRIEF DESCRIPTION OF THE FIGURES

FIG. 1 illustrates the results of flow cytometry analysis of freshly dissociated CD3 LPL for the co-expression of CD4 and either CD8α or CD8β. It showed that double-positive CD4CD8αα T lymphocytes (DP8α) are frequent in the lamina propria of healthy colon mucosa and that their CD4CD8αα phenotype remains stable in culture.

(a) Representative frequencies of CD4 T cells co-expressing CD8α or CD8β among the CD3 IEL from 12 donors; ***P<0.001 (paired t-test).

(b) Frequencies of CD4CD8αα^{low} cells among the CD3 or CD3CD4 LPL (n=18).

(c) Stable co-expression of CD4 and CD8α but not CD8β, by a cell line (representative of 4) obtained from FACS-sorted DP8α colonic LPL, after several transfers in culture.

FIG. 2 illustrates flow cytometry analysis of Freshly dissociated IEL for the co-expression of CD4 and either CD8α or CD8β. It showed the presence of double-positive CD4CD8 T lymphocytes among the intraepithelial lymphocytes (IEL) of healthy colon mucosa. Representative dot-plots (FIG. 2 a) and frequencies (FIG. 2 b) of CD4 T cells co-expressing the CD8α or CD8β among CD3 IEL from 8 donors; **P<0.01 (paired t-test).

FIG. 3 illustrates the Regulatory phenotype of DP8α LPL, as assessed by flow cytometry.

Comparison of the phenotypes of autologous DP8α and CD4 LPL lines (representative of 3 pairs).

FIG. 4 illustrates the Regulatory phenotype of DP8α LPL, as assessed by flow cytometry.

Phenotypes of DP8α and CD4 lymphocytes among CD3 LPL freshly dissociated from healthy colon mucosa (representative of 3).

FIG. 5 illustrates the Regulatory phenotype of DP8α LPL, as assessed by flow cytometry.

Expression of transcription factors by a DP8α cell line (representative of 3).

FIG. 6 illustrates the Regulatory phenotype and cytokine profile of DP8α LPL, as assessed by flow cytometry.

(a) Intracellular staining for TNF-α, IFN-γ and IL-2 in a DP8α LPL line (representative of 3) stimulated for 5 h with an anti-CD3 antibody in the presence of BFA.

(b) IL-10 in the supernatants of DP8α LPL lines (n=3) activated (black histograms) or not (white histograms) by an anti-CD3 antibody for 48 h as measured by ELISA.).

FIG. 7 illustrates the markers and cytokine profile of DP8α LPL.

(a) Expression of transcription factors by DP8α LPL freshly dissociated from healthy colonic lamina propria samples, as assessed by flow cytometry.

(b) cytokines produced by DP8α LPL freshly dissociated from colonic lamina propria samples, assessed as in FIG. 6a.

(c) IL-10 in the supernatants of DP8α and CD4 LPL lines (n=3) activated by an anti-CD3 antibody for 48 h as measured by ELISA; ***P<0.001 (t-test).

FIG. 8 illustrates the regulatory functions of DP8α LPL lines as assessed by flow cytometry.

(a) DP8α LPL block the maturation of iDC induced by activated CD4 lymphocytes, as shown by the inhibition of CD83 and CD86 upregulation, and this inhibition is partially neutralised by anti-CTLA-4 and anti-LFA-1 antibodies. Immature DC were incubated for 5 days with activated CD4 lymphocytes (expressing CD40L) in the presence or absence of DP8α LPL and anti-CTLA4 or anti-LFA-1 antibodies. The CD83 and CD86 expression levels were measured on gated CD3 negative cells: representative histograms and median for the CD83 and CD86 RFI (Relative Fluorescence Intensity); (n=6, two experiments performed with three cell lines); ***P<0.001, **P<0.01 and *P<0.05 (paired t-test).

(b) Inhibition of the proliferative response of CD4 PBL by the DP8α LPL lines C114, C139 and C192, as measured by CFSE dilution. CD4 PBL sorted from healthy donor PBMC were stimulated with anti-CD3 and anti-CD28 in the presence or absence of DP8α LPL for 5 days at a ratio of 1:1: representative cytometry data and histograms showing the mean CFSE dilution in CD8negative lymphocytes: unstimulated (white histograms), stimulated (black histograms) and stimulated in the presence of DP8α LPL (grey histograms) (n=18: six experiments done with three cell lines); ***P<0.001 (paired t-test).

(c) Percent inhibition of CD4 lymphocyte proliferation by DP8α LPL at the indicated E:T ratios.

(d) Percent suppression of CD4 lymphocyte proliferation by DP8α LPL at a ratio of 1:1 in the presence or absence of anti-IL-10 or anti-TGF-βR blocking antibodies; ***P<0.001 (paired t-test).

FIG. 9 illustrates that DP8α LPL lines specifically respond to the gut commensal bacterium Faecalibacterium prausnitzii (F).

(a) Flow cytometry analysis of the proliferative responses (VPD dilution) of DP8α LPL lines (n=4) after 3 days of co-culture with allogeneic monocytes loaded overnight with F, or not loaded in the presence or absence of an anti-MHC class-II antibody or of an irrelevant antibody, or loaded with B, L or E: mean percentage of VPD low cells (n=6: two independent experiments performed with three DP8α LPL lines; **P<0.01 (paired t-test).

(b) Flow cytometry analysis of the IFN-γ and IL-10 responses of DP8α LPL lines (n=4) after 6 h of stimulation by allogeneic monocytes loaded overnight by F (1:5) or not loaded in the presence or absence of anti MHC class-II antibody or an irrelevant antibody, or loaded with B, L or E: mean % of DP8α cells producing IFN-γ and/or IL-10 and percentages of cells secreting IL-10 or IFN-γ in independent experiments (2 to 7 experiments performed with four cell lines: C114: black circles; C139: white circles; C192: white squares and C140: black squares); ***P<0.001 (paired t-test).

FIG. 10 illustrates that F-reactive CD4CD8αα^{low} (DP8α) T lymphocytes are present in the blood of healthy subjects.

(a) Flow cytometry analysis of the frequencies of DP8α T lymphocytes in the blood of 18 donors: percentages of DP8α lymphocytes among the CD3 and the CD3CD4 PBMC.

(b) Proliferative responses (% VPD dilution) of DP8α peripheral blood T cells to F in the presence or absence of an anti-MHC class-II antibody or an irrelevant antibody, and to B, L and E. PBMC were cultured for 5 days with the antibody and/or indicated bacteria at a bacterium:PBMC ratio 1:1: percentage of VPD^{low} DP8α T cells in the PBMC from several donors (n=18); ***P<0.001 and *P<0.01 (paired t-test).

(c) Percent VPD dilution in paired DP8α and CD4 T cells among PBMC co-cultured with F for 5d; ***P<0.001 (paired t-test).

FIG. 11 illustrates that cell lines obtained from CD4CD8α^{low} PBL (DP8α PBL) (n=3) are phenotypically and functionally similar to regulatory DP8α LPL lines.

(a) The DP8α PBL lines had a Treg phenotype, in contrast with CD4 PBL lines.

(b) Representative inhibition of CD4 T lymphocyte proliferation by the DP8α PBL lines in vitro (n=6: two experiments performed with three cell lines).

(c) IL-10 secretion by the DP8α PBL lines upon stimulation with anti-CD3 antibody as measured by ELISA.

(d) Representative inhibition of DC maturation by DP8α PBL lines (n=6: 2 experiments performed with 3 cell lines).

(e) IL-10 and IFN-γ responses of the DP8α PBL lines to monocytes loaded or not with F, B, L or E: representative percentages of IFN-γ and IL-10 secreting cells (three experiments performed with 2 two to three cell lines). ***P<0.001 (paired t-test).

FIG. 12 illustrates that frequencies of DP8α LPL and PBL are lower in samples from IBD patients compared with healthy mucosa and blood samples, respectively, and that responses to F of PBL from patients with active IBD decrease compared with PBL from healthy donors and IBD patients in remission.

(a) Representative frequencies of DP8α lymphocytes among CD3 LPL freshly dissociated from the inflamed mucosa of IBD patients (black circles, n=14) and healthy colon mucosa from CC patients (white circles, n=18) *P<0.05 (t-test).

(b) Representative frequencies of DP8α PBL in IBD patients (black circles, n=30) and healthy donors (white circles, n=18) ***P<0.001 (t-test).

(c) Flow cytometry analysis of the proliferative response (% VPD^{low} cells) of DP8α PBMC from healthy donors (HD) (n=21) or IBD patients (n=20), after 5 d of co-culture with F; ***P<0.001 (t-test).

(d) Percent F-responder (VPD^{low}) DP8α cells among PBMCs from L1 and L3 Crohn's disease patients in remission (R) and with active disease (A); *P<0.05 (Mann-Whitney test).

### EXAMPLE 1

### Materials and Methods

Cell Isolation and Cell Line Generation.

PBMC were obtained from patients with inflammatory bowel disease (IBD) (Table I) and healthy subjects. Normal and inflamed colonic mucosa were obtained, respectively, from patients undergoing surgery for colon cancer who did not undergo radiotherapy or chemotherapy, and from patients undergoing surgery for IBD (Crohn's disease or ulcerative colitis) (Table II).

TABLE 1
Characteristics of the donors used to study DP8α LPL

| | Crohn's | Ulcerative | | Healthy |
|---|---|---|---|---|
| | Disease | Colitis | Total IBD | Mucosa |
| N | 10 | 3 | 13 | 18 |
| Mean age (range) | 34.7 (28-39) | 29.3 (51-27) | 35.2 (27-51) | 69 (44-82) |
| Male (%) | 30 | 33.3 | | 30 |

TABLE 2
Characteristics of the donors used to study DP8 α PBL

| | Crohn's disease | | | | Ulcerative colitis | | | | | Healthy |
|---|---|---|---|---|---|---|---|---|---|---|
| | Active | | Remission | Total | Active | Remission | Total | Total IBD | | donors |
| N | 17 | | 5 | 22 | 8 | 0 | 8 | 30 | | 17 |
| Mean age (range) | 34.8 | (19-61) | 34 (24-55) | 34.6 | 33.6 | 0 | 33.6 | 35.6 | (19-61) | 48.7 (26-58) |

| | | | | | | | | |
|---|---|---|---|---|---|---|---|---|
| Male (%) | 70.5 | 40 | 63.6 | 12.5 | 0 | 12.5 | 50 | 88 |

Montreal classification

| | | | |
|---|---|---|---|
| L1 | 8 | 0 | 8 |
| L2 | 3 | 1 | 4 |
| L3 | 6 | 4 | 10 |

Treatment

| | | | | | | | | | | | | |
|---|---|---|---|---|---|---|---|---|---|---|---|---|
| 5-ASA | 2 | (11.8%) | 0 | 2 | (9.1%) | 1 | (12.5%) | 0 | 1 | (12.5%) | 3 | (10%) |
| Corticosteroids | 5 | (29.4%) | 0 | 5 | (22.7%) | 5 | (62.5%) | 0 | 5 | (62.5%) | 10 | (33.3%) |
| Azathioprine | 12 | (70.6%) | 3 (60%) | 15 | (68.2%) | 2 | (25%) | 0 | 2 | (25%) | 17 | (56.6%) |
| Infliximab | 5 | (29.4%) | 0 | 5 | (22.7%) | 1 | (12.5%) | 0 | 1 | (12.5%) | 6 | (20%) |

| | | | | | | | | | | |
|---|---|---|---|---|---|---|---|---|---|---|
| Adalimumab | 1 | (5.9%) | 1 (40%) | 2 | (9.1%) | 0 | 0 | 0 | 2 | (6.6%) |
| Antibiotics | 1 | (5.9%) | 0 | 1 | (0.5%) | 0 | 0 | 0 | 1 | (3.3%) |

| | | | | | | | | | | | |
|---|---|---|---|---|---|---|---|---|---|---|---|
| None | 0 | 2 | 2 | (9.1%) | 1 | (12.5%) | 0 | 1 | (12.5%) | 3 | (10%) |

Monocytes and PBL were obtained from healthy donor blood by elutriation (DTC platform, CHU, Nantes, France). Normal colonic mucosa was obtained from surgically resected tissue, taken at approximately 10 cm downstream of the tumour. For normal mucosa, the lamina propria was separated from the epithelium after incubation in EDTA buffer (30 minutes) and then minced into 1-mm² fragments and washed with RPMI containing penicillin (10%) and gentamycin (0.1 mg/ml Sigma-Aldrich). Tissue fragments were digested with collagenase and DNAse (2 mg/ml each Sigma-Aldrich), with shaking at 37° C. Mucus and large debris were removed by filtration through a 40-μm cell strainer (BD). Viable cells were obtained by Ficoll gradient centrifugation. This study was approved by the local medical ethics committee. All the patients signed informed consent forms. For cell line generation, CD3CD4 and CD3CD4CD8α^{low} LPL and PBL were isolated, by sorting on a FACS-Aria (Becton Dickinson). T cell lines were generated by stimulations with PHA, irradiated feeder cells and IL-2, as described by Fonteneau et al. (J Immunol 159, 2831-2839, 1997). For suppression assays of T cell proliferation, CD4 T cells were isolated from PBMC using magnetic beads (130-045-101 Miltenyi). Immature DC were obtained from monocytes cultured for 5 days at 2×10⁶ cells/ml with 80 ng/ml IL4 and 90 ng/ml GM-CSF (AbCys) in RPMI 1640 supplemented with L-glutamine, penicillin-streptomycin (10 μg/ml) (GIBCO®) and 10% SVF (PAA). Dendritic cell maturation was induced by co-culture with activated CD4 lymphocyte or CD4 cell lines expressing the CD40L.

Flow Cytometry.

The CD8α LPL subpopulation was identified by co-staining with PerCP-conjugated anti-CD3 (345766), FITC-conjugated anti-CD4 (ref. 555346) and APC-conjugated anti-CD8α (ref. 555369) or anti-CD8α antibodies (ref. 641058). The DP8α PBL were identified by co-staining with anti-CD3, anti-CD4 and anti-CD8α antibodies (as above), and by gating on CD3CD4 cells expressing lower amounts of CD8α than CD8αβ T cells do. The following antibodies were used to phenotype the CD8α and CD4 LPL and PBL (ex vivo and as cell lines): phycoerythrin (PE)-conjugated anti-CD25 (ref. 555432), anti-CTLA4 (ref. 555853), anti-LAGS (ref. 514782), anti-CD40L (ref. 335853), anti-LFA1 (555384), anti-LFA3 (555921), anti-ICAM1 (555511), anti-TCRαβ (ref. 555548), anti-CD103 (ref. 550260), anti-FOXP3 (ref. 17477771), anti-GATA3 (ref. 560574), anti-TBET (ref. 125825), anti-RORγc (ref. 12698880), (all purchased from Becton Dickinson) anti-CD80 (ref. IM2729U), anti-CD83 (ref. IM2218U), anti-CD86 (ref. IM1976U), anti-GITR (ref. FAB689), (purchased from Beckman), and PE-conjugated anti-human TCR Vβ chains (Immunotech Beckman Coulter). The following colour- and isotype-matched control antibodies were used to confirm the staining specificities: APC-conjugated mouse IgG1 (ref. 555751), PE-conjugated-mouse IgG1 (ref. 555749), and PE-conjugated-mouse-IgG2ak (ref. 555574) (all purchased from Becton Dickinson). Single-stained beads (Comp Beads Becton Dickinson) for each fluorochrome, were used for compensation settings. Cells (2×10⁵) were stained in PB/0.1% BSA containing antibodies for 30 min at 4° C. in the dark. The cells were washed and 10⁵ cells were acquired in the viable cell gate, on a FACScalibur or a Canto II flow cytometer and analysed using Diva or CellQuest softwares (BD). The data were further analyzed with FlowJo software (Tree Star).

Suppressive Assays: Inhibition of CD4 Proliferation and DC Maturation.

Freshly sorted CD4 PBL (5×10⁴) were incubated with 5 μM CFSE (Invitrogen) in PBS containing 0.1% BSA for 15 min, washed and then stimulated with anti-CD3/anti-CD28 activation beads (Miltenyi) at a 1:1 ratio, in the presence or absence of DP8α Treg or CD4 LPL lines at the indicated effector:target (E:T) ratios. The proliferation of target CD4 T cells was assessed by flow cytometry analysis of CFSE dilution among CD8-negative T cells, on day 5 in the presence and or absence of DP8α LPL, or of CD4LPL and in the presence or absence of anti-IL-10 or anti-TGF-β antibodies. The CD4 LPL lines were cultured with immature allogeneic DC (1:1 ratio) for 2 to 3 d in the presence or absence of DP8α cell lines and anti-CTLA-4 or anti-LFA-1 antibodies. The cells were stained with APC-conjugated anti-CD3 (ref. 555335) and PE-conjugated anti-CD80 (ref. IM2729U), PE-conjugated anti-CD83 (ref. IM2218U), or PE-conjugated anti-CD86 (ref. IM1976U) antibodies. CD3 negative cells were analysed by flow cytometry to determine the level of expression of CD80, CD83 and CD86.

Intracellular Cytokine Assays.

PBMC were incubated 6 h with plate-bound 0.1 μg/ml plate-bound CD3 antibody (OKT3 eBioscience) or with bacteria at a 1:1 ratio. T cell lines (LPL and PBL) were incubated 6 h with anti-CD3 (as above) or at a 1:1 ratio with a mixture of allogeneic monocytes previously incubated overnight with the different bacteria at a 5:1 ratio. To prevent cytokine secretion, 10 μg/ml brefeldin A (Sigma-Aldrich) was added for the last 6 h of stimulation. Peripheral blood T cells and cell lines were stained with PerCP-conjugated anti-CD3, FITC-conjugated anti-CD4 and APC-conjugated anti-CD8α (as described above). The cells were then fixed for 10 min in PBS/4% paraformaldehyde (Sigma-Aldrich) and washed. Cytokine-specific antibodies were then added for 30 min at room temperature. Reagent dilutions and washes were performed with PBS containing 0.1% BSA and 0.1% saponin (Sigma-Aldrich). Cytokine secretion was assessed by flow cytometry in CD3CD4CD8α^{low} and CD3CD4CD8α^{neg} PBMC and in DP8α or CD4-positive cell lines. In some experiments Vβ labelling and intracellular labelling were further analyzed among cytokine-labelled DP8α cell lines. The following antibodies were used: PE-conjugated anti-IL-2 (ref. 559334), anti-IL-4 (ref. 554486), anti-IL-5 (ref. 554395), anti-IL-10 (ref. 562400), anti-TNF-α (ref. 554418) and anti-IL-22 (ref. 515303) and APC-conjugated anti-IL-13 (ref. 554571), anti-IFN-γ (ref. 554551) and anti-IL-17 (ref. 51717871). For blocking experiments, anti HLA class II ascites (clone 206 produced in our laboratory) and an irrelevant mouse IgG were used.

T Cell Cytokine Production by ELISA.

T cells (10⁵ in 200 μl) were stimulated with plate bound anti-CD3 antibody (OKT3, eBioscience) at 0.1 μg/ml for 2 d. The levels of IL-10 in the supernatants were measured by ELISA (R&D Systems).

Bacterial Cultures.

Faecalibacterium prausnitzii A2-165 (F) was grown for 20 h at 37° C. in LYBHI medium (brainheart infusion medium supplemented with 0.5% yeast extract (Difco), cellobiose (1 mg/ml; SigmaAldrich), maltose (1 mg/ml; Sigma-Aldrich), and cysteine (0.5 mg/ml; Merck) in an anaerobic chamber. Bacteroides thetaiotaomicron VPI-5482 (B) and Lactobacillus casei (ATCC 393) (L) were grown for 20 h at 37° C. in an anaerobic chamber in Wilkins-Chalgren medium (33 g/L; Oxoid) and LYBHI medium, respectively. Escherichia coli K12 (E) was grown for 20 h at 37° C. with agitation (80 r.p.m) in Luria-Bertani medium (20 g/L; Invitrogen). The supernatant and pellet for each bacterial strain were obtained by centrifugation at 1700 g at 4° C. for 15 min.

T Lymphocyte Proliferation Assays to Bacteria.

Lymphocytes (PBMC or cell lines) were labelled for 15 min incubation at 37° C. in the dark with 1 μM VPD (BD Bioscience) or CFSE as described above (in one case) in PBS containing 0.1% BSA. The cells were washed twice in medium containing 10% FBS. F, B, L and E were sonicated for 15 min at high speed and then co-cultured with VPD labeled PBMC at 1:1 ratio, or with monocytes overnight (at a ratio 5:1) in presence of gentamycin (0.1 mg/ml). Monocytes loaded with bacteria or left unloaded were washed and mixed with VPD-labelled cells (1 to 1.5×10⁵) at a 1:5 ratio. After 3 to 7 d, the proliferation of T cells was assessed by flow cytometry analysis of the VPD (or CFSE) dilution in CD3CD4CD8a^{low} and CD3CD4CD8a^{neg} PBMC or in CD4− or CD8α positive T cells. The HLA class II dependency was analysed by adding a specific antibody every 48 h or an irrelevant mouse IgG.

Statistical Analysis

Statistical analysis was performed with the GraphPad Prism version 5.0 (GraphPad software). Paired and unpaired t-tests and the Mann-Whitney test were used, as indicated in the figure legends. Differences were considered significant at P<0.05.

### EXAMPLE 2

### Double-Positive CD4CD8αα T Lymphocytes (DP8α) are Frequent in the Human Colonic Lamina Propria

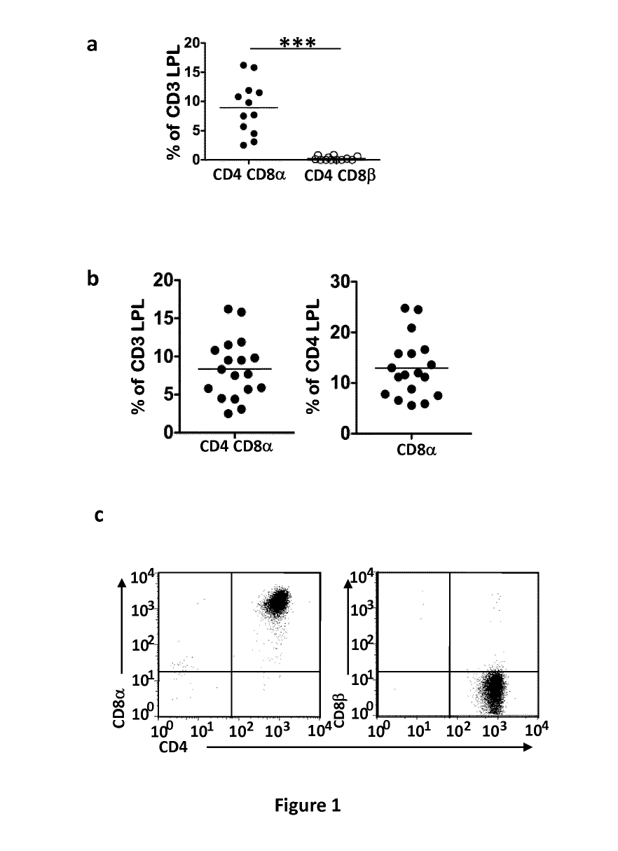

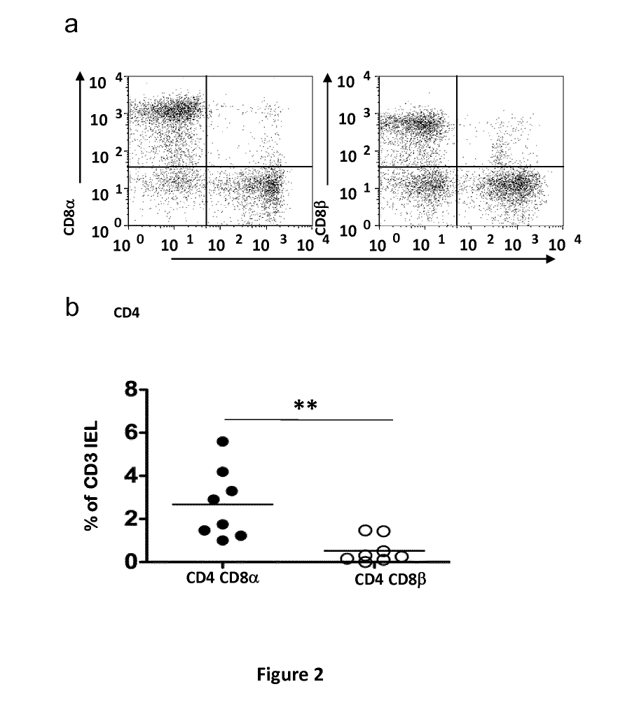

The co-expression of CD4 and CD8α or CD8β by T cells isolated from the epithelium or LP of healthy colonic mucosa from colon carcinoma (CC) patients were analysed. A significant fraction of CD3 lamina propria lymphocytes (LPL) co-expressed CD4 and CD8α but not CD8β (FIG. 1a). The CD8α level was variable and lower than that on CD8 αβ T cells (data not shown). CD4CD8αα^{low} LPL, thereafter referred to as DP8α LPL, were then quantified (FIG. 1b). They made up as a mean 8.5% (range 3.1-16.2) of CD3 and 13.3% (range 5.9-24.8) of CD4 LPL. In the epithelium, smaller T cell fractions co-expressed CD4 and CD8α (mean 2.4% range 0.7-5.6) and some of these expressed high levels of CD8α or CD8β (FIG. 2), likely corresponding to the CD4CD8αβ colonic IEL subset previously described (Sarrabayrouse et al. Int J Cancer 128, 2923-2932, 2011).

Unsorted and FACS-sorted DP8α, CD4 and CD8αβ LPL lines were then derived to evaluate the phenotypic stability and polyclonality of DP8α LPL. At any time during culture, the CD8 expression by sorted DP8α cell lines was conserved (FIG. 1c and data not shown). Among unsorted LPL lines (n=3) the DP8α lymphocytes expressed as many distinct Vβ chains (10 to 14) as their CD8 and CD4 counterparts (11-12 and 9-16, respectively), but shared more Vβ chains with the latter (60-91%) than the former (36-50%) (data not shown). Therefore, DP8α T cells are abundant in the LP of the human colonic mucosa where they represent a polyclonal T cell subset phenotypically distinct from the CD4 LPL.

### EXAMPLE 3

### DP8α Colonic LPL Lines Exhibit a Treg Phenotype and Functions

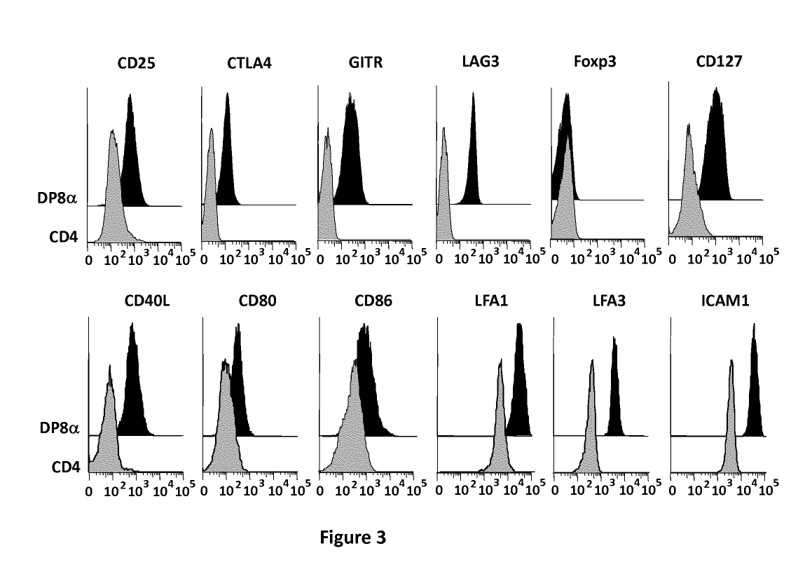

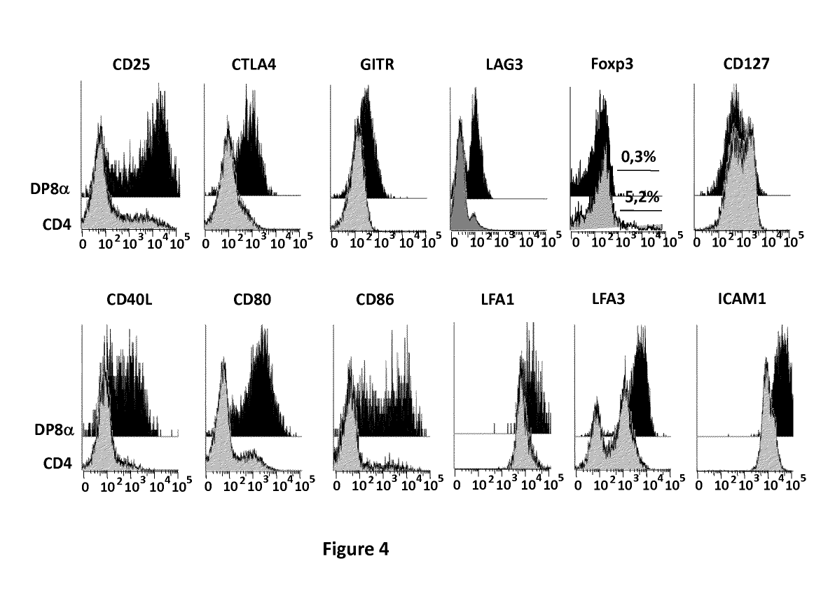

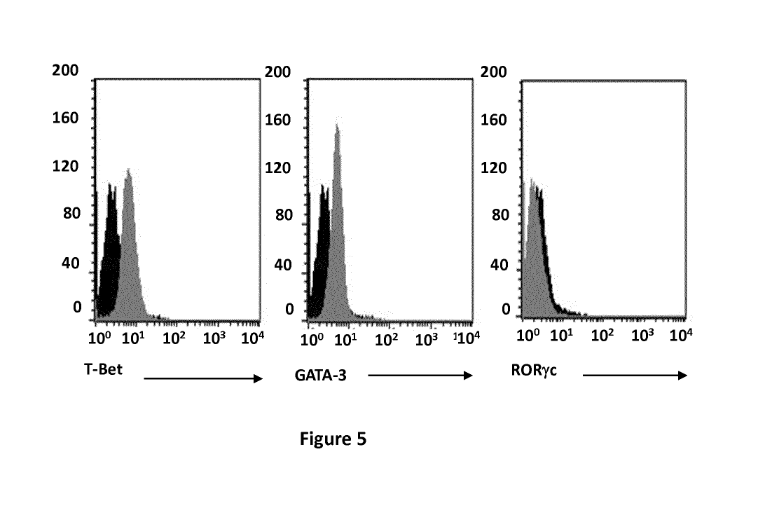

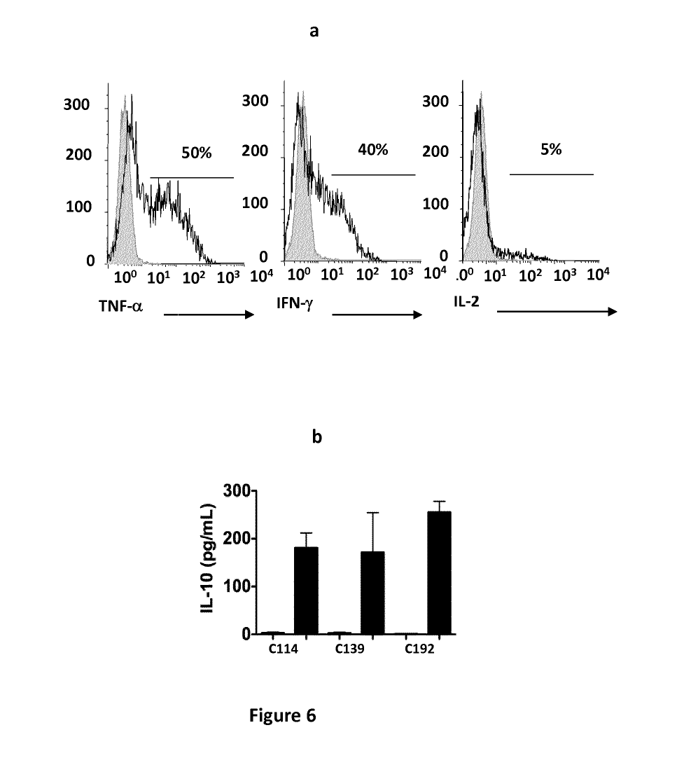

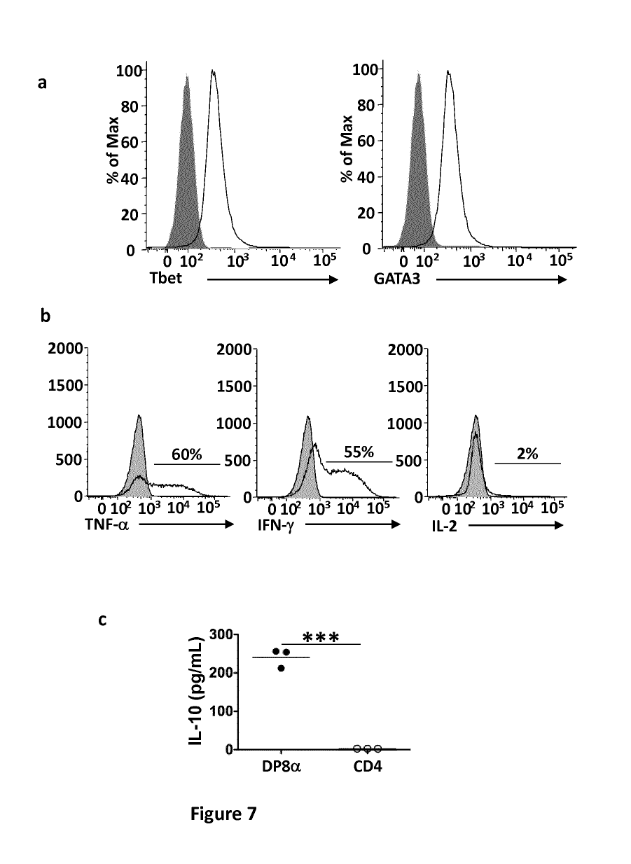

It was then asked whether DP8α human colonic LPL have a Treg phenotype, compared with their CD4 homologues. As illustrated by one DP8α LPL line representative of 3 (FIG. 3), DP8α LPL differed from their autologous CD4 counterparts by the expression or over-expression of Foxp3-Treg markers (e.g., CD25, CTLA4, GITR, LAG-3), activation and co-stimulation markers (e.g., CD80, CD86, CD40L) and adhesion markers (e.g., LFA-1, LFA3 and ICAM-1). However, in contrast to Foxp3-Treg, DP8α LPL expressed the IL-7R (CD127) and lacked Foxp3. The major fraction of ex-vivo DP8α LPL exhibited an identical phenotype with the exception of a low IL-7R (CD127) expression (FIG. 4). The DP8α LPL lines and DP8α LPL expressed Tbet and Gata3 (FIG. 5 and FIG. 7a). The former also lacked RORγc (FIG. 5). Upon polyclonal activation, the DP8α cell lines secreted TNF-α and IFN-γ (approximately 50% cells), but little if any IL-2 (FIG. 6a) and no IL-4, IL-5, IL-13, IL-17 or IL-22 (data not shown). Ex vivo, DP8α LPL exhibited the same cytokine profile as the DP8α cell lines (FIG. 7b). Importantly, as shown by RT-PCR (data not shown) and ELISA, activated DP8α LPL lines (FIG. 6b), but not their CD4 counterparts (FIG. 7c), secreted IL-10. Therefore, DP8α LPL exhibit cell surface markers and a cytokine profile of Treg but lack Foxp3. The regulatory potential of these cells in vitro was then addressed.

### EXAMPLE 4

### Regulatory Properties of Human DP8α LPL

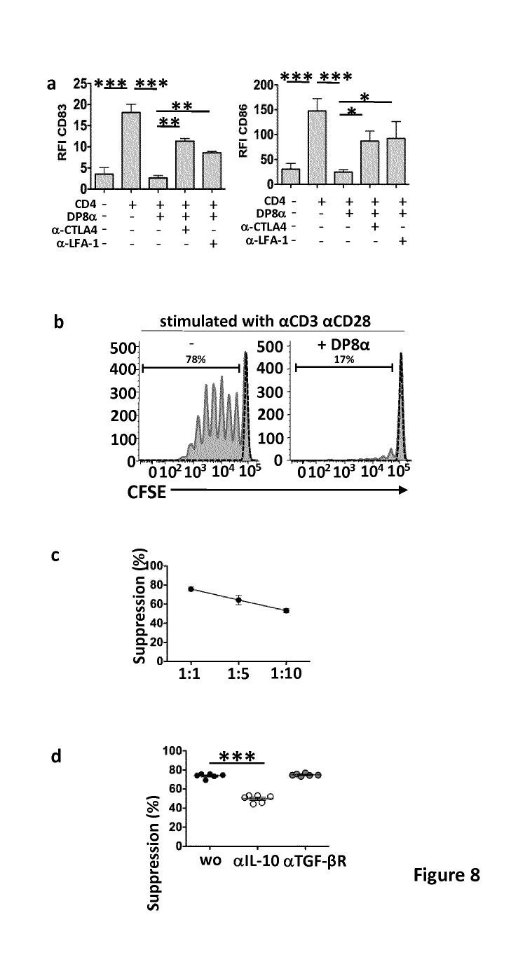

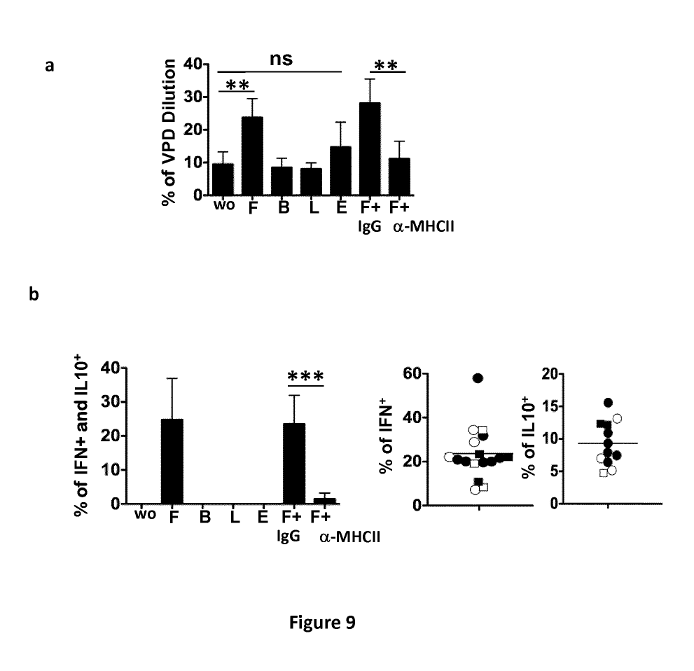

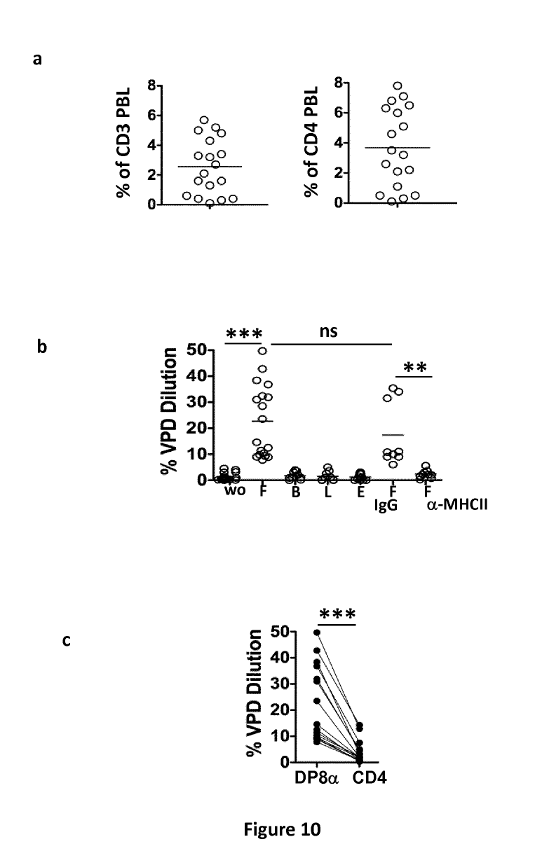

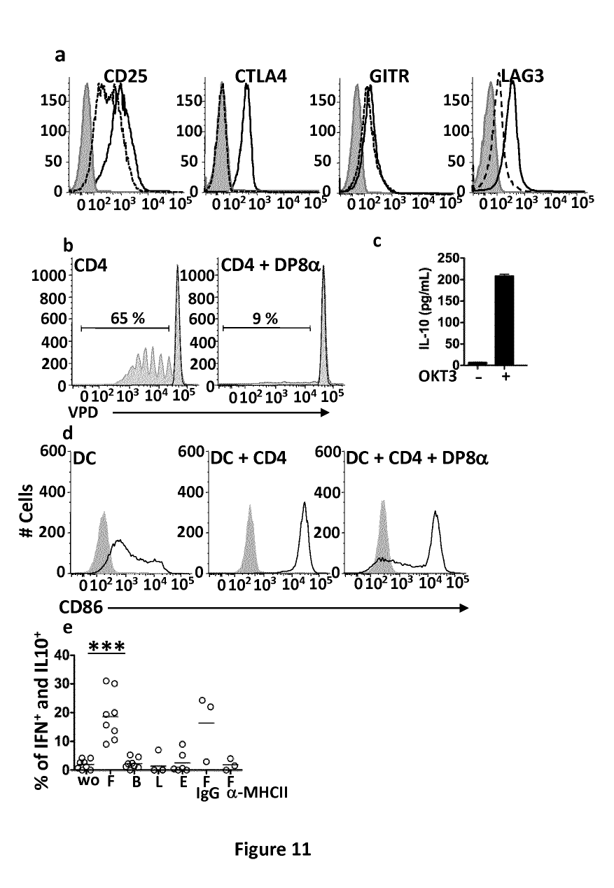

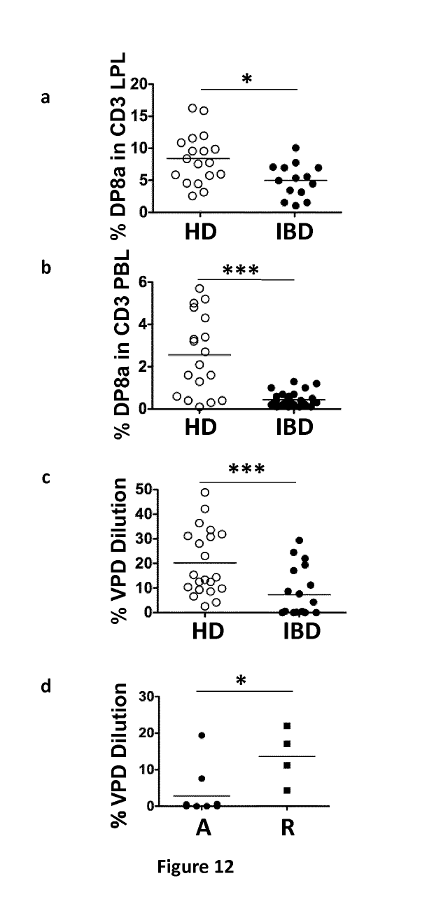

Similarly to Foxp3 Treg, the DP8α LPL lines inhibited the maturation of immature dendritic cells, as revealed by the inhibition of CD86, CD83 (FIG. 8a) and CD80 (data not shown)-up-regulation, in a CTLA-4 and LFA-1-dependent manner (FIG. 8a). The DP8α LPL lines also inhibited CD4 T cell proliferation induced by anti-CD3 and anti-CD28 antibody at all effector-target ratios used (FIGS. 8b and 8c) and this inhibition was partially blocked by anti-IL10 but not by anti-TGF-βR-antibody (FIG. 8d). Importantly, the CD4 LPL lines lacked these regulatory properties (data not shown). During these assays it was observed that DP8α LPL lines proliferated upon CD3 activation in vitro, in contrast to what has been reported for Foxp3 Treg (data not shown).

### EXAMPLE 5

### DP8α LPL Lines Specifically React with a Gut Commensal Bacteria

Bacteria belonging to the Clostridium IV and XIV groups are outstanding inducers of Foxp3 Treg in the mouse colonic LP and some of these Treg express TCR specific for Clostridium antigens. Therefore, the microbiota reactivity of DP8α LPL was assessed using four bacteria strains: Faecalibacterium prausnitzii (F), a major human gut bacterium of the Clostridium IV group, that is present at a decreased level in the faeces of IBD patients and was shown to induce IL-10 expression by PBMC (Sokol et al. Proc Natl Acad Sci USA 105, 16731-16736, 2008), Bacteroides thetaiotaomicron (B) and Lactobacillus casei (L), which may promote Foxp3 Treg differentiation/expansion in mice (Honda, K. & Littman, D. R., Annu Rev Immunol 30, 759-795, 2012), and Escherichia coli (E), a potential pathobiont. The DP8α LPL lines did not proliferate or secrete cytokines when incubated with these bacteria alone (data not shown). In contrast, they responded systematically to monocytes loaded with F, but not to monocytes alone or monocytes loaded with B, L and E bacteria, as shown by their proliferation (FIG. 9a). Both responses were suppressed by an anti-MHC class-II antibody but not by an irrelevant antibody. The DP8α LPL lines also responded specifically to F-loaded monocyte in a MHC class-II-dependent manner secreting IFN-γ and IL-10 within 6 hours of stimulation. Surprisingly, up to 20 to 60% cells in the LPL lines were F-reactive (cytokine positive) in this assay (FIG. 9b). In contrast, the CD4 LPL lines made no proliferative or cytokine response to bacteria-loaded monocytes (data not shown). In the DP8α LPL lines, the F-reactive cells expressed 2 to 5 Vβ chains out of 21 tested (data not shown). Importantly, CD4 LPL expressing these Vβ chains did not respond to F-loaded monocytes (data not shown), ruling out the possibility that these responses were due to superantigens. Therefore, the DP8α LPL lines, but not their CD4 counterparts, contained a high proportion of cells that reacted specifically to Faecalibacterium prausnitzii, in an APC- and MHC class-II-dependent fashion.

### EXAMPLE 6

### DP8α Regulatory T Cells are Present Amongst PBL

It was found that CD4CD8αα^{low} T cells represented as a mean 2.5% (range 0.1-5.8%) of CD3 PBL and 3.7% (range 0.1-7.8%) of CD4 PBL in healthy donors (FIG. 10a). To determine whether these cells could be Treg originating from the colon mucosa, the expression of regulatory markers (CTLA4, CD25, GITR and LAGS), and their reactivity to F were assessed. Ex vivo, most CD4CD8αα^{low} PBL lacked these markers (data not shown). Nonetheless, a significant proportion of these cells proliferated within 5 days of culture with F (mean % VPD dilution 22.7, range 7.8-49.7) in a MHC class-II dependant manner, but not, or at much lower levels, with other bacteria (FIG. 10b). Compared with CD4CD8αα^{low} PBL, CD4 PBL yielded much lower proliferative responses to F (mean % VPD dilution: 3.8 range 0.3-14.3) (FIG. 10c) and did not respond to E (data not shown). To further determine whether CD4CD8αα^{low} PBL could be Treg, CD4CD8αα^{low} (DP8α) PBL lines were derived from three healthy subjects. One cell line was derived from CD3 PBL sorted ex vivo for the co-expression of CD4 and of low levels of CD8α. Two additional DP8α cell lines and one CD4 cell line were obtained using a similar sort from PBMC cultured 5 days with F. Under both conditions the first sort yielded CD4CD8αα^{low} cells with a purity of 60-70%. Pure DP8α and CD4 PBL lines (data not shown) were obtained after a second sort. Unexpectedly, the DP8α cell lines expressed high levels of CD25, CTLA-4, GITR, and LAG-3 in culture (FIG. 11a). In contrast, the CD4 PBL line lacked CTLA-4 and expressed CD25 and LAG-3 at lower levels than its DP8α counterparts (FIG. 11a). The DP8α PBL lines also secreted IL-10 (FIG. 11c) and inhibited the proliferation of CD4 lymphocytes and the maturation of dendritic cells (FIGS. 11b and 11d). Between 9 and 31% of the cells in these lines secreted IFN-γ and/or IL-10 in response to monocytes loaded with F but not to monocytes loaded with the other bacteria, in a MHC class-II-dependent manner (FIG. Ile), but did not secrete IL-2 or IL-4 (data not shown). In contrast, the CD4 PBL line did not secrete IL-10, lacked regulatory function and failed to secrete IL-10, IFN-g, IL-2 and IL-4 in response to F-loaded monocytes (data not shown). Therefore, the majority of CD4CD8αα^{low} PBL appear to be circulating DP8α Treg, because they acquired regulatory markers and functions in culture, and because a proportion of these cells reacted to F, while their CD4 counterparts lacked these properties.

### EXAMPLE 7

### Decreased Frequency of DP8α LPL and PBL and Decreased F-Reactivity of DP8α PBL in Patients with Inflammatory Bowel Diseases Compared with Healthy Donors

Because the inventors have shown that high proportions of DP8α LPL and PBL were specific for F and since it was previously shown that this bacterium is present at reduced levels in the gut microbiota of patients with IBD, the inventors asked whether the frequencies of DP8α cells were altered in IBD patients compared with non-IBD donors (see Tables 1 and 2 above for patient and healthy subject characteristics). The mean frequency of DP8α cells among CD3 LPL appeared to be lower in the inflamed colon mucosa of IBD patients than in the healthy mucosa of patients with colorectal cancer (respectively 4.8%, range 0.9-9.9 and 8.5%, range 3.1-16) (FIG. 12a). Moreover, the mean fraction of CD4CD8αα^{low} (DP8α) lymphocytes among CD3 PBL was lower for IBD patients than for healthy donors (respectively 0.4%, range 0.1-1.3 and 2.5%, range 0.1-5.7) (FIG. 12b).

The reduced level of Faecalibacterium prausnitzii in the gut microbiota of Crohn's disease patients previously reported might affect the production of F-specific Treg. To address this hypothesis, the inventors compared the proliferative responses to F of DP8α PBL from healthy subjects and IBD patients. DP8α PBL from healthy donors proliferated systematically upon PBMC co-culture with F (mean % VPD^{low} DP8α PBL at day 5: 20.2, range 2.6 to 48.9) (FIG. 12c), but not when cultured alone or with E (data not shown). A similar proliferation was observed among the PBMC from 9 out of 20 IBD patients. In the remaining patients, DP8α PBL did not proliferate at all (n=5) or proliferated only non-specifically, i.e. as much to E and/or in the absence of bacteria as to F (n=6). As a result, the mean specific response to F of DP8α PBL from IBD patients (IBD) was lower (% VPD^{low} cells at day 5: 6.3, range: 0 to 29.4) than in healthy donors (HD) (FIG. 12c). Because Crohn's disease patients with ileal involvement (L1 and L3 stages of the Montreal classification) exhibit the clearest deficit in F in the gut microbiota, the inventors assessed whether remission in these patients was associated with a restored response of DP8α PBL to F.

DP8α PBL from L1 and L3 patients in remission (n=4) proliferated specifically to F (mean 13.6, range 4.3-22). In contrast, such a response was significantly less frequent in patients with active disease (2 out of 10) mean 2.8, range 0-19.4 (FIG. 12d).

### EXAMPLE 8

### In Vitro Induction of DP8α Regulatory T Cell Differentiation by F. prausnitzii and/or Butyrate

### Material and Methods

### (i) Cells

Naive CD4 T cells are obtained from PBMC or PBL using the EasySep negative selection kit for naive CD4 T cells (STEMCELL technologies).

Monocytes are isolated from PBMC using the CD14 microbeads positive selection kit of Miltenyi.

Immature dendritic cells (iDC) are prepared from monocytes (CD14+) cultured for 3 days with IL-4 (200 IU/ml) and GM-CSF (1000 IU/ml). Said cells are also called mo-iDC (monocyte-derived immature dendritic cells).

### (ii) Method of Induction of DP8a Treg

Immature DC are co-cultured for a night with thawed F. prausnitzii bacteria at 1:1 ratio in the presence or not of butyrate. The iDC are then added to naive CD4 cells for 5 days in the presence of IL-2.

Alternatively, butyrate is added later, at the same time as naive CD4 T cell.

DP8α induction is measured by a labeling with CD3-PE (phycoerythrin), CD4-FITC, CD8-APC (allophycocyanin) antibodies. The results are compared with those obtained with unstimulated CD4 T cells, CD4 T cells stimulated by iDC alone and CD4 T cells stimulated by iDC loaded with E. coli.

### Results

Results of induction are shown in Table 3 below.

TABLE 3
Differentiation induction of naive CD4 T cells

| | Buty- | Mean of DP8α | Range of DP8α |
|---|---|---|---|
| Stimulation | rate | cells (%) | cells (%) |
| Unloaded monocytes | − | 0.6 | 0.2-1.6 |
| F. prausnitzii-loaded monocytes | − | 2.9 | 1.2-4.6 |
| E. coli-loaded monocytes | − | 1.2 | 0.6-2.4 |
| Unloaded iDCs | − | 1 | 0.3-2.2 |
| F. prausnitzii -loaded mo-iDC | − | 3 | 1.5-6.9 |
| | + | 6 | 1.8-16.5 |
| E. coli -loaded mo-iDC | − | 1.7 | 0.9-2.4 |
| | + | 2 | 1.3-2.9 |

+: addition of 0.145 mM of butyrate during the loading of the monocytes or mo-iDC with F. prausnitzii

The stimulation of naive CD4 T cells for 4 to 6 days by monocytes or monocyte-derived immature dendritic cells (mo-iDC) previously cultured overnight with F prausnitzii induces the expression of low levels of CD8α on a fraction of naive CD4 T cells (mean 2.9%, range 1.2-4.6% and mean 3%, range 1.5-6.9%, respectively). In contrast, when monocytes or iDC are loaded with E. coli, very few DP8α cells appear (mean 1.2%, range 0.6-2.4% and mean 1.7%, range 0.9-2.4%, respectively).

The impact of short chain fatty acids (SOFA) on the induction of DP8α lymphocytes is then tested: butyrate, acetate and propionate produced by the gut microbiota. The addition of butyrate (0.145 mM) to iDC loaded with F. prausnitzii strongly increases the fraction of naive CD4 T cells that acquire the DP8α phenotype (mean 6%, range 1.8-16.5%) in culture. In contrast, propionate and acetate have no or few effect on the frequency of DP8α cells.

By sorting DP8α lymphocytes induced by F. prausnitzii-loaded iDC and butyrate with a FACS Aria, DP8α cell lines are obtained. These cells lines have also regulatory properties: expression of regulatory markers (CD25, CTLA-4, GITR and LAG-3), secretion of IL-10 and inhibition of CD4 T cell proliferation.

Besides, F. prausnitzii induces the maturation of mo-iDC (obtained by a 3 day culture with IL-4 and GM-CSF) and induces the secretion by these cells of IL-10. Moreover, in the presence of butyrate, the maturation of iDC induced by F. prausnitzii is inhibited, as shown by decreased expression of CD80 and CD83 (but not of CD86 and HLA-DR), and the secretion of IL-10 is increased.

These data indicate that DP8α Treg may be induced in vitro by a short culture of naive CD4 T lymphocytes with monocyte or mo-iDC co-cultured overnight with F. prausnitzii and that butyrate may enhance this induction, potentially through the inhibition of DC maturation and/or through the enhancement of IL-10 secretion by iDC. Alternatively, butyrate may stimulate the expansion of DP8α Treg induced by F. prausnitzii.

1. An in vitro method of determining if a subject is afflicted with an inflammatory bowel disease comprising:
(a) determining the number and/or concentration and/or proportion of T regulatory lymphocytes with a CD4⁺ CD8αα^{low} Foxp3^{neg} phenotype which are specific for F prausnitzii in a biological sample from the subject; and
(b) deducting from the result(s) of step (a), if the subject is afflicted with an inflammatory bowel disease.

2. An in vitro method of prognosing outcome of an inflammatory bowel disease in a patient suffering from an inflammatory bowel disease comprising a step of determining the number and/or concentration and/or proportion of T regulatory lymphocytes with a CD4⁺ CD8αα^{low} Foxp3^{neg} phenotype which are specific for F prausnitzii in a biological sample from the patient.

3. The in vitro method of prognosing outcome of an inflammatory bowel disease according to claim 2, wherein said method comprises the following steps:
(a) determining the number and/or concentration and/or proportion of T regulatory lymphocytes with a CD4⁺ CD8αα^{low} Foxp3^{neg} phenotype which are specific for F prausnitzii in a biological sample from the patient; and
(b) deducing from the result of step a) the prognosis of said patient.

4. An in vitro method of predicting whether a subject is at risk of developing an inflammatory bowel disease, said method comprising:
a) determining the number and/or concentration and/or proportion of T regulatory lymphocytes with a CD4+ CD8αα^{low} Foxp3^{neg} phenotype which are specific for F prausnitzii in a biological sample from the subject;
b) predicting from the result of step (a) if the subject is at risk of suffering from an inflammatory bowel disease, wherein a low value at step (a), relative to a reference value for a sample of the same nature, indicates that said subject is at risk of developing an inflammatory bowel disease.

5. An in vitro method of monitoring the efficacy of a preventive or curative treatment of an inflammatory bowel disease, the method comprises a step of monitoring the number and/or concentration and/or proportion of T regulatory lymphocytes with a CD4⁺ CD8αα^{low} Foxp3^{neg} phenotype which are specific for F prausnitzii in a biological sample from the subject during the treatment.

6. The method according to claim 1, wherein the inflammatory bowel disease is the Crohn's disease.

7. The method according to claim 1, wherein the biological sample is chosen from the group consisting of a colonic mucosa sample, a biopsy sample from the gastrointestinal tract, a colonic biopsy sample, a small intestine biopsy sample, a blood sample, a blood fraction, including peripheral blood mononuclear cells (PBMC) and peripheral blood lymphocytes (PBL).

8-10. (canceled)

11. A method for activating and/or inducing the proliferation of T regulatory lymphocytes with a CD4⁺ CD8αα^{low} Foxp3^{neg} phenotype which are specific for F. prausnitzii in a patient in need thereof, comprising a step of administering to said patient a composition comprising:
(i) a F. prausnitzii strain; and/or
(ii) a live-attenuated F. prausnitzii strain; and/or
(iii) a killed F. prausnitzii strain; and/or
(iv) at least one fragment of at least one of strains (i), (ii) and (iii); and/or
(v) antigen-presenting cells (APCs) loaded with at least one of (i), (ii), (iii) and (iv).

12. The method according to claim 11, wherein the antigen-presenting cells are antigen-presenting cells obtained from the subject to be treated.

13. A kit for diagnosing, prognosing and/or predicting the risk of developing an IBD, and/or for monitoring the efficacy of a treatment of an IBD, wherein said kit comprises means for determining the number and/or concentration and/or proportion of T regulatory lymphocytes with a CD4⁺ CD8αα^{low} Foxp3^{neg} phenotype specific for F prausnitzii.

14. A kit for treating or preventing an inflammatory bowel disease (IBD) comprising:
a packaging material;
a known amount of:
(i) a population of isolated T regulatory lymphocytes, with a CD4⁺ CD8αα^{low} Foxp3^{neg} phenotype, which are specific for F. prausnitzii, or a pharmaceutical composition thereof; and/or
(ii) an isolated F. prausnitzii strain, or a pharmaceutical composition thereof; and/or
(iii) an isolated live-attenuated F. prausnitzii strain, or a pharmaceutical composition thereof; and/or
(iiii) an isolated killed F. prausnitzii strain, or a pharmaceutical composition thereof; and/or
(v) at least one fragment of at least one of strains (ii), (iii) and (iv), or a pharmaceutical composition thereof; and/or
(vi) antigen-presenting cells (APCs) loaded with at least one of (i), (ii), (iii) and (iv); loaded with at least one of the compounds (ii), (iii), (iv) and (v), or a pharmaceutical composition thereof.

15. A process for obtaining T regulatory lymphocytes with a CD4⁺ CD8αα^{low} Foxp3^{neg} phenotype which are specific for F. prausnitzii, said process comprising:
a) culturing APCs (Antigen-presenting Cells) in the presence of F. prausnitzii, thereby obtaining F. prausnitzii loaded APCs, and
b) culturing naïve CD4 T cells in the presence of
the F. prausnitzii loaded APCs obtained in step a),
thereby obtaining T regulatory lymphocytes with a CD4⁺ CD8αα^{low} Foxp3^{neg} phenotype which are specific for F. prausnitzii.

16. A method for treating or preventing an inflammatory bowel disease in a patient in need thereof, comprising a step of administering to said patient a therapeutically effective amount of isolated T regulatory lymphocytes with a CD4⁺ CD8αα^{low} Foxp3^{neg} phenotype which are specific for F. prausnitzii.

17. The method according to claim 16, wherein the T regulatory lymphocytes with a CD4⁺ CD8αα^{low} Foxp3^{neg} phenotype which are specific for F. prausnitzii are autologous lymphocytes obtained from the subject to be treated.

18. The method according to claim 16, wherein the T regulatory lymphocytes with a CD4⁺ CD8αα^{low} Foxp3^{neg} phenotype which are specific for F. prausnitzii are primary culture cells or established cell lines.

19. The in vitro method according to claim 1, wherein said method comprises between step (a) and step (b): a step (a1) of comparing the result of step (a) with i) a control standard value corresponding to the number and/or concentration and/or proportion of these T regulatory lymphocytes typically found in a biological sample of the same nature from a healthy subject, and/or ii) a control standard value corresponding to the number and/or concentration and/or proportion of these T regulatory lymphocytes typically found in a biological sample of the same nature from a patient suffering from an inflammatory bowel disease; and wherein step b) comprises deducting from the result(s) of step (a) and/or step (a1), if the subject is afflicted with an inflammatory bowel disease.

20. The method according to claim 19, wherein the inflammatory bowel disease is Crohn's disease.

21. The method according to claim 19, wherein the biological sample is selected from the group consisting of a colonic mucosa sample, a biopsy sample from the gastrointestinal tract, a colonic biopsy sample, a small intestine biopsy sample, a blood sample, a blood fraction, including peripheral blood mononuclear cells (PBMC) and peripheral blood lymphocytes (PBL).

22. The method according to claim 2, wherein the inflammatory bowel disease is Crohn's disease and/or wherein the biological sample is selected from the group consisting of a colonic mucosa sample, a biopsy sample from the gastrointestinal tract, a colonic biopsy sample, a small intestine biopsy sample, a blood sample, a blood fraction, including peripheral blood mononuclear cells (PBMC) and peripheral blood lymphocytes (PBL).

23. The method according to claim 4, wherein the inflammatory bowel disease is Crohn's disease and/or wherein the biological sample is selected from the group consisting of a colonic mucosa sample, a biopsy sample from the gastrointestinal tract, a colonic biopsy sample, a small intestine biopsy sample, a blood sample, a blood fraction, including peripheral blood mononuclear cells (PBMC) and peripheral blood lymphocytes (PBL).

24. The method according to claim 5, wherein the inflammatory bowel disease is Crohn's disease and/or wherein the biological sample is selected from the group consisting of a colonic mucosa sample, a biopsy sample from the gastrointestinal tract, a colonic biopsy sample, a small intestine biopsy sample, a blood sample, a blood fraction, including peripheral blood mononuclear cells (PBMC) and peripheral blood lymphocytes (PBL).

25. The process according to claim 15, wherein the APCs are cultured in step a) in the presence of F. prausnitzii and butyrate.

26. The process according to claim 15, wherein the naïve CD4 T cells are cultured in step b) in the presence of IL-2 and/or butyrate.

27. The kit according to claim 14, wherein said kit comprises a label or package insert contained with said packaging material indicating that the “drugs” (i) to (vi), or pharmaceutical compositions thereof, are effective in the prevention and/or treatment of an IBD, in a subject suffering from an IBD or at risk of developing an IBD.

28. The kit according to claim 14, wherein the IBD is Crohn's disease.
